(19) United States
(12) Patent Application Publication
(10) Pub. No.: US 2024/0009181 A1
(43) Pub. Date: Jan. 11, 2024

(54) METHOD FOR THE TREATMENT AND PROPHYLAXIS OF SUBSTANCE-RELATED AND/OR ADDICTIVE DISORDERS INCLUDING SUBSTANCE/MEDICATION-INDUCED DEPRESSIVE DISORDERS, BIPOLAR AND RELATED DISORDERS, AND ANXIETY DISORDERS AND COMPOSITIONS USEFUL FOR THE SAME

(71) Applicants: Jason Eric Schiffman, Los Angeles, CA (US); Kenneth B. Kashkin, Sparta, NJ (US)

(72) Inventors: Jason Eric Schiffman, Los Angeles, CA (US); Kenneth B. Kashkin, Sparta, NJ (US)

(21) Appl. No.: 17/811,437

(22) Filed: Jul. 8, 2022

(51) Int. Cl. A61K 31/4709; A61K 9/20; A61K 47/59; A61P 25/36

(52) U.S. Cl. CPC A61K 31/4709; A61K 9/2077; A61K 47/593; A61K 9/2086; A61P 25/36

(57) ABSTRACT

Pharmaceutical compositions and dosage forms containing low doses of aripiprazole (Formula (I)) or its derivatives/congeners/analogues (e.g., brexpiprazole or cariprazine), and topiramate (Formula (II)) are provided. A method of treatment and/or prophylaxis of substance-related and/or addictive disorders by administering a combination of aripiprazole, or its congeners/analogues/derivatives (e.g., brexpiprazole or cariprazine), and topiramate is also provided. The aripiprazole is generally administered in immediate release in a daily dose in the range of 1.5 mg to 6 mg, in combination with a controlled release formulation daily dose of topiramate of 23 to 92 mg. Additionally provided is a method of treating a patient wherein a composition containing topiramate and aripiprazole or its derivatives/congeners/analogues is administered for purposes of treatment, and/or prophylaxis of a substance-induced depressive, bipolar or anxiety disorder and other psychiatric conditions. Methods of administration of low doses of topiramate and aripiprazole or its congeners/analogues (e.g., brexpiprazole or cariprazine) are also provided.

### TECHNICAL FIELD

The present disclosure relates to methods of treatment and prophylaxis of Substance-related and/or Addictive Disorders, including substance/medication-induced depressive disorders, bipolar and related disorders, and anxiety disorders, and pharmaceutical compositions useful for the same.

### BACKGROUND

As defined by The Diagnostic and Statistical Manual of Mental Disorders, 5th Edition (“DSM-5”), Substance-related and Addictive Disorders include Substance Related Disorders and Non-Substance Related (Addictive) Disorders. The prevalence of substance-related and/or addictive disorders is on the rise globally and, especially, in the United States, where opioid use disorders, in particular, are considered so prevalent and serious as to be termed an “epidemic”. There are serious and significant social, economic and health consequences of substance-related and/or addictive disorders. Strategies for treating substance-related and/or addictive disorders have included community-based, psychotherapeutic, and pharmacologic approaches. The difficulty of treating Substance-related and/or addictive disorders through psychotherapeutic and behavioral interventions alone has continued to necessitate the use of pharmacotherapies. However, there are currently only a limited number of effective pharmacotherapies for substance-related and/or addictive disorders and the United States Food and Drug Administration (FDA) has approved only a few drugs for a small subset of these disorders. Additionally, despite the overwhelming co-occurrence and mutual reinforcement of substance use disorders and mental health disorders, there are currently no FDA approved pharmacotherapies indicated specifically for any of the substance/medication-induced mental disorders. Of particular concern, because of the “opioid epidemic”, are the limited number and effectiveness of approved pharmacotherapies for opioid use disorders. The three FDA approved pharmacotherapies are all opioid receptor ligands—functioning either as agonist, partial agonist, or antagonist. The FDA approved pharmacotherapies for opioid dependence are 1) methadone, a mu-opioid receptor agonist, 2) various formulations of buprenorphine (e.g. Suboxone), a mu opioid receptor partial agonist, and 3) naltrexone (e.g, Vivitrol), a mu opioid receptor antagonist. These are often termed Medication-assisted Treatments (MAT), and labeling for these drugs requires they be used in combination with counseling and behavioral therapies. There are currently no non-opioid based pharmacotherapies approved for opioid use disorders.

The current opioid-based pharmacotherapies have been beneficial but have serious limitations and adverse effects, in great part because of the very fact that they act on the opioid system itself through activation or blockade of opioid receptors. As drugs with agonist action acting at the mu opioid receptor, methadone and buprenorphine have multiple adverse effects including intoxication, tolerance, withdrawal, endocrine dysfunction, and potentially fatal overdose, particularly if combined with other central nervous system depressants. Furthermore, both methadone and buprenorphine are notoriously difficult to discontinue due to significant and protracted withdrawal symptoms, and thus once initiated many patients never discontinue these medications leading to lifelong exposure to their adverse effects. Additionally, many patients treated with methadone and buprenorphine may be less likely to participate in or benefit from non-pharmacologic interventions for addictive disorders due to the opioid effects on motivation and emotional processing. Long-acting naltrexone (Vivitrol) is a mu opioid receptor antagonist and thus free of the opioid agonist effects described above. While this eliminates the adverse effects associated with methadone and buprenorphine, naltrexone is associated with its own adverse effects, particularly depression, a symptom to which this patient population is already particularly vulnerable, which can lead to discontinuation of treatment and an increased risk for relapse. Additionally, long-acting naltrexone has been associated with an increased risk of fatal overdose upon relapse to opioid use after discontinuation. Noteworthy is that response to treatment with naltrexone is disappointing; complete abstinence in response to treatment with opioid blockade by naltrexone (defined as opioid-free over 19 weeks of treatment) in the clinical study cited in FDA approved labeling was sustained by only 36% of subjects treated with naltrexone vs. 23% given placebo. Perhaps the most important limitation of all current pharmacotherapies for opioid use disorder is the fact that as opioid receptor ligands they cannot be effectively used in patients actively using opioids. Finally, because each of the three FDA approved medications for opioid use disorder act at the same receptor but with different actions, they may not be used concomitantly, limiting pharmacological treatment of opioid use disorder to monotherapy. Thus, the need to find alternative pharmacotherapies for opioid use disorders that do not directly modulate the opioid system is one of the greatest challenges and opportunities for advancement in the treatment of serious substance related and addictive disorders.

Aripiprazole is an atypical antipsychotic drug approved by the FDA in 2002 and is characterized as a serotonin-dopamine activity modulator (“SDAM”). Aripiprazole's FDA approved labeling includes the treatment of schizophrenia, bipolar mania, agitation associated with schizophrenia or bipolar mania, major depressive disorder, irritability associated with autistic disorder, and Tourette's disorder.

Topiramate is a drug with FDA approved labeling for the treatment of epilepsy and migraine. Topiramate was approved by the FDA in 1996.

Aripiprazole's demonstrated efficacy for depressive symptoms is believed to result from its activity on serotonergic and dopaminergic neurons. Additionally, both aripiprazole and topiramate modulate neurotransmission in the mesolimbic (“reward”) pathway. Aripiprazole affects mesolimbic neurotransmission through partial agonism of dopamine D2 receptors while topiramate does so through facilitation of GABA-mediated inhibitory impulses, and antagonism of AMPA and kainate glutamate receptors.

### BRIEF SUMMARY

The present application discloses an exemplary pharmaceutical composition comprising:
a first active pharmaceutical ingredient which is aripiprazole or a derivative, congener, or an analogue, or a pharmaceutically acceptable salt thereof, wherein the first active pharmaceutical ingredient is present in an amount of 0.5 to 12 mg, and
a second active pharmaceutical ingredient which is topiramate or a pharmaceutically acceptable salt thereof, wherein the second active pharmaceutical ingredient is present in an amount of 5 to 150 mg;
   wherein the pharmaceutical composition is a single dosage form.

The present application also discloses an exemplary method of treatment or prophylaxis of substance-related and/or addictive disorders, and/or substance-induced depressive disorders, bipolar and related disorders, or anxiety disorders, comprising administering to a patient: a first active pharmaceutical ingredient which is aripiprazole, or a derivative, congener, or an analogue, or a pharmaceutically acceptable salt thereof, and a second active pharmaceutical ingredient which is topiramate or a pharmaceutically acceptable salt thereof.

An exemplary pharmaceutical composition, comprises:
a first active pharmaceutical ingredient which is aripiprazole or a derivative, congener, or an analogue, or pharmaceutically acceptable salt thereof, wherein the first active pharmaceutical ingredient is present in an amount of 0.5 to 12 mg, and
a second active pharmaceutical ingredient which is topiramate or a pharmaceutically acceptable salt thereof, wherein the second active pharmaceutical ingredient is present in an amount of 20 to 95 mg.

Other embodiments and advantages of the present disclosure will be apparent from the following description, drawings and the appended claims.

### BRIEF DESCRIPTION OF THE DRAWINGS

FIG. 1 shows an exemplary embodiment of topiramate plasma concentration vs. time curve for immediate release topiramate and low dose topiramate ER.

FIG. 2 shows an exemplary embodiment of predicted aripiprazole plasma concentration vs. time curves for low dose aripiprazole with and without co-administered low dose topiramate ER.

FIG. 3 shows an exemplary embodiment of predicted aripiprazole plasma concentration vs. time curves for the median dose of aripiprazole for various FDA approved indications, and curves for low dose aripiprazole with and without co-administered low dose topiramate ER.

FIG. 4 shows an exemplary embodiment of a table with predicted percentage reduction in aripiprazole steady state plasma levels achieved by the present disclosure (co-administered low dose aripiprazole and low dose topiramate ER) relative to the steady state plasma levels of FDA recommended aripiprazole doses for various indications.

FIG. 5 shows an exemplary embodiment of the aripiprazole striatal dopamine D2 receptor occupancy percentage associated with the median doses of aripiprazole for various FDA approved indications as well as for low dose aripiprazole with and without co-administered low dose topiramate ER, as discussed herein.

FIG. 6 shows an exemplary embodiment of various binding sites of aripiprazole ordered from higher to lower affinity with associated Ki and action.

### DETAILED DESCRIPTION

Aripiprazole is an atypical antipsychotic drug, which has the chemical formula C₂₃H₂₇C₁₂N₃O₂ and the following structure as shown in Formula (I):

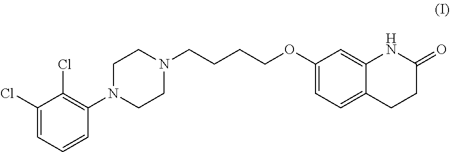 [SMILES: O=C1CCc2ccc(OCCCCN3CCN(c4cccc(Cl)c4Cl)CC3)cc2N1]

Aripiprazole has been described as a serotonin-dopamine activity modulator (“SDAM”). Aripiprazole was approved by the FDA in 2002. Aripiprazole is indicated by FDA approved labeling for the treatment of schizophrenia, bipolar mania, agitation associated with schizophrenia or bipolar mania, major depressive disorder, irritability associated with autistic disorder, and Tourette's disorder. The recommended daily dose of aripiprazole for the treatment of depressive and schizophrenic disorders ranges from 5 mg to 30 mg. The half-life of aripiprazole is about 75 hours. Aripiprazole is marketed under the brand name ABILITY®.

Topiramate is a drug with FDA approved labeling for the treatment of epilepsy and migraine. It has the chemical formula C₁₂H₂₁NO₈S and the following structure as shown in Formula (II):

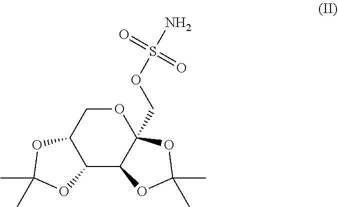

Topiramate was approved by the FDA in 1996. The recommended daily dose of topiramate for the treatment of migraine and epilepsy ranges from 100 mg to 400 mg. The half-life of topiramate is about 21 hours. Topiramate is marketed under the brand name TOPAMAX®.

Aripiprazole's demonstrated efficacy for depressive symptoms is believed to result from its activity on serotonergic and dopaminergic neurons. Additionally, both aripiprazole and topiramate modulate neurotransmission in the mesolimbic (i.e., “reward”) pathway. Aripiprazole achieves this modulation through partial agonism of dopamine D2 receptors while topiramate does so through facilitation of GABA-mediated inhibitory impulses, and antagonism of AMPA and kainate glutamate receptors.

The present disclosure provides in an exemplary embodiment, a pharmaceutical composition comprising:
a first active pharmaceutical ingredient which is aripiprazole or a derivative, congener, or an analogue, or a pharmaceutically acceptable salt thereof, wherein the first active pharmaceutical ingredient is present in an amount of 0.5 to 12 mg, and
a second active pharmaceutical ingredient which is topiramate or a pharmaceutically acceptable salt thereof, wherein the second active pharmaceutical ingredient is present in an amount of 5 to 150 mg;
   wherein the pharmaceutical composition is a single dosage form.

In an exemplary pharmaceutical composition as disclosed herein, the active pharmaceutical ingredients in the pharmaceutical composition consist of the first and second active pharmaceutical ingredients. For example, no active pharmaceutical ingredients are present in addition to aripiprazole, its derivatives/analogues/congeners, and topiramate, or their pharmaceutically acceptable salts thereof.

In another exemplary embodiment, the pharmaceutical composition is administered orally. For example, as an oral formulation.

In yet another exemplary embodiment, the pharmaceutical composition is disclosed, wherein an oral formulation comprises one or more tablets or capsules.

In an exemplary embodiment, the first and second active pharmaceutical ingredients are present in a ratio [first active pharmaceutical ingredient]:[second active pharmaceutical ingredient] of 1.5:23.

In a further exemplary embodiment is the pharmaceutical composition disclosed herein, further comprising a controlled release polymer selected from the group consisting of delayed release polymers and extended-release polymers.

In another exemplary embodiment, the controlled release polymer layer comprises one or more polymers selected from the group consisting of EUDRAGIT® NM 30D, ethylcellulose, polyvinylpyrrolidone, polylactides (PLA), polyglycolides (PGA), poly(lactide-co-glycolides) (PLGA), polyanhydrides, and polyorthoesters.

In an exemplary pharmaceutical composition disclosed herein, the composition is a bilayer tablet comprising an immediate release drug layer and one or more extended-release drug layers. For example, two extended-release drugs. In another embodiment, the pharmaceutical composition disclosed herein comprises a gel-matrix tablet.

In another exemplary embodiment, the pharmaceutical composition or dosage form is a bilayer tablet comprising an immediate release drug layer and one or more delayed release drug layers. For example, two delayed-release drugs.

In one exemplary embodiment, the immediate release drug layer disclosed herein contains aripiprazole or a derivative, congener, or an analogue, or a pharmaceutically acceptable salt thereof, and the extended-release drug layer contains topiramate or a pharmaceutically acceptable salt thereof.

In an exemplary pharmaceutical composition, the immediate release drug layer contains aripiprazole or a derivative, congener, or an analogue, or a pharmaceutically acceptable salt thereof, and the delayed release drug layer contains topiramate or a pharmaceutically acceptable salt thereof.

Brexpiprazole is an atypical antipsychotic drug having the chemical formula C₂₅H₂₇N₃O₂S and the following structure of Formula (III):

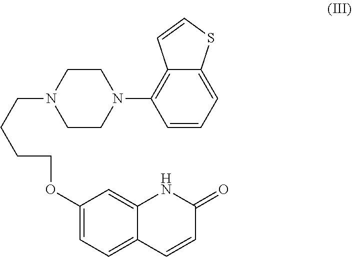 [SMILES: O=c1ccc2ccc(OCCCCN3CCN(c4cccc5sccc45)CC3)cc2[nH]1]

Brexpiprazole is a dopamine D2 receptor partial agonist and has been described as a “serotonin-dopamine activity modulator” (SDAM). Brexpiprazole was approved by the U.S. Food and Drug Administration in 2015. Brexpiprazole is prescribed for the treatment of schizophrenia, bipolar disorder, and major depressive disorder, and may be useful for treating attention deficit/hyperactivity disorder. The half-life of brexpiprazole is about 91 hours.

Brexpiprazole is marketed under the brand name REXULTI®. As used herein, “brexpiprazole” denotes brexpiprazole as well as any pharmaceutically acceptable salt thereof.

Cariprazine is also an atypical antipsychotic drug having the chemical formula C₂₁H₃₂Cl₂N₄O and the following structure of Formula (IV):

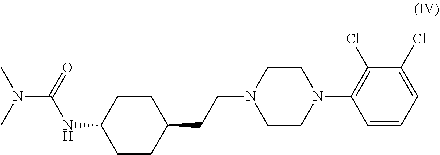 [SMILES: CN(C)C(=O)N[C@H]1CC[C@H](CCN2CCN(c3cccc(Cl)c3Cl)CC2)CC1]

Cariprazine is a dopamine D2 and D3 receptor partial agonist and has been described as a “serotonin-dopamine activity modulator” (SDAM). Cariprazine is also a partial agonist at 5-HT1A receptor and acts as an antagonist at 5-HT2B and 5-HT2A receptors. Cariprazine was approved by the U.S. Food and Drug Administration in 2015. Cariprazine is prescribed for the treatment of schizophrenia, and bipolar disorder such as bipolar mania and bipolar depression. The half-life of cariprazine is about 48 to 96 hours.

Cariprazine is marketed under the brand name VRAYLAR®. As used herein, “cariprazine” denotes cariprazine as well as any pharmaceutically acceptable salt thereof.

In another exemplary embodiment, the first active pharmaceutical ingredient comprises brexpiprazole and/or cariprazine.

In another exemplary embodiment, there is disclosed a method of treatment or prophylaxis of substance-related and/or addictive disorders, and/or substance-induced depressive disorders, bipolar and related disorders, or anxiety disorders, comprising administering to a patient: a first active pharmaceutical ingredient which is aripiprazole, or a derivative, congener, or an analogue, or a pharmaceutically acceptable salt thereof, and a second active pharmaceutical ingredient which is topiramate or a pharmaceutically acceptable salt thereof. For example, the addictive disorder is a non-substance related i.e. gambling disorder.

In one exemplary method of treatment or prophylaxis, the substance-related and/or addictive disorder is selected from the group consisting of opioid-related disorders, alcohol-related disorders, cannabis-related disorders, tobacco-related disorders, hallucinogen-related disorders, stimulant-related disorders, and sedative-, hypnotic-, anxiolytic-related disorders and gambling-related disorder; and/or
wherein the substance-related disorder is selected from the group of substance use disorders consisting of opioid use disorder, alcohol use disorder, cannabis use disorder, tobacco use disorder, stimulant use disorder, hallucinogen use disorder, and sedative, hypnotic, and anxiolytic use disorder; and/or
wherein the substance-related and/or addictive disorder is selected from the group of substance-induced disorders consisting of 1) opioid-induced depressive disorder, cannabis-induced depressive disorder, alcohol-induced depressive disorder, stimulant-induced depressive disorder, sedative-, hypnotic-, or anxiolytic-induced depressive disorder, alcohol-induced depressive disorder, and gambling-induced depressive disorder, 2) opioid-induced bipolar and related disorder, cannabis-induced bipolar and related disorder, alcohol-induced bipolar and related disorder, stimulant-induced bipolar and related disorder, sedative-, hypnotic-, or anxiolytic-induced bipolar and related disorders, alcohol-induced bipolar and related disorder, and 3) opioid-induced anxiety disorder, cannabis-induced anxiety disorder, stimulant-induced anxiety disorder, sedative-, hypnotic-, or anxiolytic-induced anxiety disorder, and alcohol-induced anxiety disorder; and/or
wherein the substance-related and/or addictive disorder is a depressive disorder, bipolar and related disorder, or anxiety disorder associated with substance-induced intoxication or withdrawal, wherein the substance is selected from the group consisting of opioids, alcohol, cannabis, tobacco, stimulants, hallucinogens, sedatives, hypnotics, and anxiolytics; and/or wherein the substance related disorder is opioid use disorder and/or the substance-induced disorder is opioid-induced depressive disorder, bipolar and related disorder, or anxiety disorder and an opioid receptor ligand is concurrently administered or used illicitly.

For example, the stimulant in the stimulant-related or stimulant-use disorder is amphetamine or cocaine.

In an exemplary method of treatment or prophylaxis, the treatment or prophylaxis for substance-related and/or addictive disorders, and/or substance-induced depressive disorders, bipolar and related disorders, or anxiety disorders, results in an increased retention in treatment and/or a reduction in relapse after treatment or prevents the occurrence of such disorders and/or obviates the need for additional treatments or therapies; and/or wherein the treatment initiation is concurrent with substance use, intoxication, or withdrawal.

In another exemplary embodiment is the method of treatment or prophylaxis discussed herein, wherein naltrexone has been previously administered, or is currently being administered, and/or has resulted in an adverse event or side-effect.

For example, the adverse event or side-effect is depression (e.g. substance-induced depression, the substance being naltrexone).

In yet another exemplary method of treatment or prophylaxis, prophylaxis prevents opioid use disorder when opioid medications are administered for analgesia.

In an exemplary method of treatment or prophylaxis, prophylaxis prevents anxiolytic use disorder when anxiolytics are administered for treatment of anxiety disorders.

In another exemplary method of treatment or prophylaxis, the treatment or prophylaxis for substance-related and/or addictive disorders, and/or substance-induced depressive disorders, bipolar and related disorders, or anxiety disorders is effective in treating or preventing the disorders at steady state aripiprazole plasma levels of less than or equal to 70 ng/ml and steady state topiramate plasma levels of less than or equal to 6 micrograms/ml at which 1) aripiprazole high-affinity targets dopamine D2, D2L, D3 receptors and serotonin 5HT2B, 5-HT1A receptors are known to be preferentially bound by aripiprazole compared to low affinity targets, 2) the D2 receptor occupancy is known to be reduced compared to occupancy at plasma levels required for treatment of current FDA approved therapeutic indications, and 3) topiramate plasma levels are below those required for treatment of current FDA approved therapeutic indications known to be associated with intolerable adverse events. In one aspect, the low steady state aripiprazole plasma levels result in high-affinity target binding such that the desired physiological response is produced, without producing the D2 receptor-induced, or lower affinity receptor-induced, adverse effects. In another aspect, steady state aripiprazole plasma levels are achieved by administering low doses of aripiprazole (e.g., 1.5-6 mg, including 1.5, 3, 4.5, and 6 mg), for example compared to the higher doses of aripiprazole (e.g., greater than 6 mg, such as 7-30 mg) currently administered to treat other indications or diseases. In an alternative aspect, steady state aripiprazole plasma levels are achieved upon administering low doses of aripiprazole (e.g., 1.5-6 mg, including 1.5, 3, 4.5, and 6 mg) in combination with topiramate selected from any one of 23, 46, 69, and 92 mg, which produce the desired physiological effect with lower probability of intolerable adverse events known to occur at the higher doses of topiramate (e.g., 100-400 mg) currently administered to treat other indications or diseases, according to the disclosure herein. For example, these may be administered in a single dosage form.

In another exemplary method of treatment or prophylaxis, the treatment or prophylaxis for substance-related and/or addictive disorders, and/or substance-induced depressive disorders, bipolar and related disorders, or anxiety disorders is effective in treating or preventing the disorders at low steady state aripiprazole plasma levels in which aripiprazole dopamine D2 receptor occupancy percentage is lower than at the steady state aripiprazole levels associated with the FDA approved dosing ranges for treatment of schizophrenia, bipolar disorder, and major depressive disorder.

In an exemplary method of treatment or prophylaxis, the treatment or prophylaxis for substance-related and/or addictive disorders, and/or substance-induced depressive disorders, bipolar and related disorders, or anxiety disorders is effective in treating or preventing the disorders while minimizing drug-induced akathisia, extrapyramidal symptoms, nausea, weight gain, sedation or difficulty in word-finding.

In another exemplary method of treatment or prophylaxis, the aripiprazole or a derivative, congener, or an analogue, or a pharmaceutically acceptable salt thereof is administered in an amount of 0.5 mg to 12 mg, and topiramate or a pharmaceutically acceptable salt thereof is administered in an amount of 5 mg to 150 mg.

In yet another exemplary method of treatment or prophylaxis, the aripiprazole or a derivative, congener, or an analogue, or a pharmaceutically acceptable salt thereof is administered continually over a significant period of time in a daily dose of 1.5, 3, 4.5, or 6 mg and, in combination therewith, a daily dose of 23, 46, 69, or 92 mg topiramate or a pharmaceutically acceptable salt thereof. For example, 1.5 mg aripiprazole, or its derivative, congener, or an analogue, or a pharmaceutically acceptable salt thereof and 23 mg topiramate or a pharmaceutically acceptable salt thereof. A further example, 3 mg aripiprazole, or its derivative, congener, or an analogue, or a pharmaceutically acceptable salt thereof and 46 mg topiramate or a pharmaceutically acceptable salt thereof. Another example, 4.5 mg aripiprazole, or its derivative, congener, or an analogue, or a pharmaceutically acceptable salt thereof and 69 mg topiramate or a pharmaceutically acceptable salt thereof. In an example, 6 mg aripiprazole, or its derivative, congener, or an analogue, or a pharmaceutically acceptable salt thereof and 92 mg topiramate or a pharmaceutically acceptable salt thereof.

In one exemplary method of treatment or prophylaxis disclosed herein, the aripiprazole or a derivative, congener, or an analogue, or a pharmaceutically acceptable salt thereof is administered continually over a significant period of time in an escalating dose regimen of 1.5 mg aripiprazole per day in combination with 23 mg topiramate per day for one week, followed by 3 mg aripiprazole per day in combination with 46 mg topiramate per day, and followed, if there is an insufficient response, by an increase of 1.5 mg aripiprazole per day in combination with an increase of 23 mg topiramate per day every two weeks up to a maximum dose of 6 mg aripiprazole per day in combination with 92 mg topiramate per day. An insufficient response would be known and understood by one of ordinary skill in the art.

In another exemplary embodiment, the pharmaceutical composition disclosed herein comprises:
a first active pharmaceutical ingredient which is aripiprazole or a derivative, congener, or an analogue, or pharmaceutically acceptable salt thereof, wherein the first active pharmaceutical ingredient is present in an amount of 0.5 to 12 mg, and
a second active pharmaceutical ingredient which is topiramate or a pharmaceutically acceptable salt thereof, wherein the second active pharmaceutical ingredient is present in an amount of 20 to 95 mg.

In an exemplary embodiment, the pharmaceutical composition comprises:
a first active pharmaceutical ingredient which is aripiprazole or a derivative, congener, or an analogue, or pharmaceutically acceptable salt thereof, wherein the first active pharmaceutical ingredient is present in an amount of 0.5 to 12 mg, and
a second active pharmaceutical ingredient which is topiramate or a pharmaceutically acceptable salt thereof, wherein the second active pharmaceutical ingredient is present in an amount of 5 to 10 mg.

In an exemplary embodiment, the pharmaceutical composition or dosage form is a bilayer tablet comprising two extended-release layers or two delayed release layers.

In an exemplary method, the disorder includes an impulsive symptom of a substance related, or non-substance related, disorder, and wherein the substance is selected from the group consisting of opioids, alcohol, cannabis, tobacco, hallucinogens, stimulants, sedatives, hypnotics, and anxiolytics, and the non-substance is gambling. For example, the disorder is substance abuse or a substance dependence, wherein the substance is selected from the group consisting of opioids, alcohol, cannabis, tobacco, stimulants, hallucinogens, sedatives, hypnotics, and anxiolytics.

In another exemplary method, the substance related disorder is an opioid related disorder and treatment initiation is concurrent with opioid use, intoxication or withdrawal.

In yet a further exemplary method, the substance related disorder is opioid use disorder and/or the substance-induced disorder is opioid-induced depressive disorder, bipolar and related disorder, or anxiety disorder and treatment with an opioid receptor ligand (agonist, partial agonist, or antagonist) has resulted in inadequate response to treatment, and/or the administered opioid receptor ligand should or must be decreased or stopped.

In another exemplary method of treatment or prophylaxis disclosed herein, the prophylaxis prevents progression of opioid use to an opioid use disorder or prevents progression from mild opioid use disorder to moderate or severe opioid use disorder.

In yet another exemplary method of treatment or prophylaxis, the aripiprazole is administered continually over a significant period of time in a daily dose of 3 mg aripiprazole and, in combination therewith, a daily dose of 46 mg topiramate.

In a further exemplary method of treatment or prophylaxis, the aripiprazole is administered continually over a significant period of time in a daily dose of 4.5 mg aripiprazole and, in combination therewith, a daily dose of 69 mg topiramate.

In an exemplary method of treatment or prophylaxis, the aripiprazole is administered continually over a significant period of time in a daily dose of 6 mg aripiprazole and, in combination therewith, a daily dose of 92 mg topiramate.

Described herein are embodiments of a pharmaceutical composition containing a first active pharmaceutical ingredient selected from aripiprazole (or a derivative, congener or analogue, e.g., brexpiprazole or cariprazine), and a second active pharmaceutical ingredient which is topiramate, or a pharmaceutically acceptable salt thereof.

Also described herein are embodiments of a method for treating substance-related and/or addictive disorders involving administering to a patient a pharmaceutical composition or dosage form comprising a first active pharmaceutical ingredient selected from aripiprazole (or a derivative, congener or analogue, e.g., brexpiprazole or cariprazine) and a second active pharmaceutical ingredient which is topiramate.

Also described herein are embodiments of a method for treating substance-induced depressive disorders, bipolar and related disorders, or anxiety disorders involving administering to a patient: a pharmaceutical composition or dosage form containing a first active pharmaceutical ingredient selected from aripiprazole (or a derivative, congener or analogue, e.g., brexpiprazole or cariprazine) and a second active pharmaceutical ingredient which is topiramate.

Accordingly, in a first embodiment, the present disclosure is directed to a method for treating substance-related and/or addictive disorders in a subject, including substance use disorders and substance-induced disorders such as substance-induced depressive disorders, bipolar and related disorders, or anxiety disorders, by administering continually over a significant period of time a daily dose of aripiprazole in the range of 1.5 mg to 6 mg and in combination therewith a daily amount of topiramate in the range of 23 mg to 92 mg. Generally, the combination of these active agents is administered orally. The agents may be administered at different times of day, with the aripiprazole administered earlier in the day and the topiramate administered later in the day, in the afternoon or evening. Normally, however, the two agents are administered simultaneously using one or more dosage forms that provide for immediate release of the aripiprazole and controlled release of the topiramate. In an exemplary embodiment, the aripiprazole and topiramate are administered in a single dosage form that provides for immediate release of the aripiprazole and both delayed and sustained release of the topiramate.

In another embodiment, the disclosure is directed to a method for treating substance-related and/or addictive disorders in a subject, including substance use disorders and substance-induced disorders such as substance-induced depressive disorders, bipolar and related disorders, or anxiety disorders, by administering continually over a significant period of time a daily dose of 1.5 mg aripiprazole and, in combination therewith, a daily dose of 23 mg topiramate.

In another embodiment, the disclosure is directed to a method for treating substance-related and/or addictive disorders in a subject, including substance use disorders and substance-induced disorders such as substance-induced depressive disorders, bipolar and related disorders, or anxiety disorders, by administering continually over a significant period of time a daily dose of 3 mg aripiprazole and, in combination therewith, a daily dose of 46 mg topiramate.

In another embodiment, the disclosure is directed to a method for treating substance-related and/or addictive disorders in a subject, including substance use disorders and substance-induced disorders such as substance-induced depressive disorders, bipolar and related disorders, or anxiety disorders, by administering continually over a significant period of time a daily dose of 4.5 mg aripiprazole and, in combination therewith, a daily dose of 69 mg topiramate.

In another embodiment, the disclosure is directed to a method for treating substance-related and/or addictive disorders in a subject, including substance use disorders and substance-induced disorders such as substance-induced depressive disorders, bipolar and related disorders, or anxiety disorders, by administering continually over a significant period of time a daily dose of 6 mg aripiprazole and, in combination therewith, a daily dose of 92 mg topiramate.

In another embodiment, the disclosure is directed to a method for treating substance-related and/or addictive disorders in a subject, including substance use disorders and substance-induced disorders such as substance-induced depressive disorders, bipolar and related disorders, or anxiety disorders, by administering continually over a significant period of time an escalating dose regimen of 1.5 mg aripiprazole per day in combination with 23 mg topiramate per day for one week, followed by 3 mg aripiprazole per day in combination with 46 mg topiramate per day, and followed, if there is an insufficient response, by an increase of 1.5 mg aripiprazole per day in combination with an increase of 23 mg topiramate per day every two weeks up to a maximum dose of 6 mg aripiprazole per day in combination with 92 mg topiramate per day.

In a further embodiment, a packaged pharmaceutical preparation is provided that contains a composition of the disclosure in a sealed container, with instructions for administration, typically self-administration, of the composition. Generally, the packaged preparation contains a plurality of orally administrable unit dosage forms, with, preferably, each individual dosage form in a separate sealed housing, e.g., as in a blister pack. For example, multiple tablets may be housed in a single dosage form.

It is believed that through their direct action on the mesolimbic pathway and other limbic pathways, compositions described herein which contain both aripiprazole (or a derivative, congener or analogue, e.g. brexpiprazole or cariprazine) and topiramate synergistically decrease cravings for substances (and activities such as those in Gambling Disorder, a non-substance related addictive disorder) and attenuate symptoms of substance withdrawal (a substance-induced disorder). In a further exemplary embodiment, known derivative, congener or analogues of aripiprazole, and topiramate, and their pharmaceutically acceptable salts are encompassed.

Their mechanism of action also, simultaneously, alleviates or otherwise improves the mood- and anxiety-related symptoms associated with substance use disorders, including Opioid Use Disorder, Alcohol Use Disorder, Stimulant Use Disorder, Sedative, Tobacco Use Disorder, Hypnotic, or Anxiolytic Use Disorder, and Cannabis Use Disorder. These compositions also target the mood symptoms and anxiety symptoms associated with substance/medication-induced mental disorders such as Opioid-Induced Depressive Disorder, Cannabis-Induced Depressive Disorder, Amphetamine (or other stimulant)-Induced Depressive Disorder, Cocaine-Induced Depressive Disorder, Sedative-, Hypnotic-, or Anxiolytic-Induced Depressive Disorder, and Alcohol-Induced Depressive Disorder, as well as Opioid-Induced Anxiety Disorder, Cannabis-Induced Anxiety Disorder, Amphetamine (or other stimulant)-Induced Anxiety Disorder, Cocaine-Induced Anxiety Disorder, Sedative-, Hypnotic-, or Anxiolytic-Induced Anxiety Disorder, and Alcohol-Induced Anxiety Disorder, Opioid-Induced Bipolar and Related Disorder, Cannabis-Induced Bipolar and Related Disorder, Amphetamine (or other stimulant)-Induced Bipolar and Related Disorder, Cocaine-Induced Bipolar and Related Disorder, Sedative-, Hypnotic-, or Anxiolytic-Induced Bipolar and Related Disorder, and Alcohol-Induced Bipolar and Related Disorder.

It is well established that mood and anxiety symptoms are both primary causes and consequences of Substance-related and/or addictive disorders and are nearly universally present among individuals with these conditions. Thus, administration of compositions described herein containing aripiprazole combined with topiramate powerfully treat Substance-related and/or addictive disorders and Substance/Medication-Induced Depressive Disorders, Bipolar and Related Disorders, and Anxiety Disorders by attenuating both cravings and mood and anxiety symptoms simultaneously and thereby inhibiting the mutually reinforcing relationship between the perpetuating factors of addiction and co-occurring mood and anxiety symptoms. Additionally, unlike nearly all other antidepressants, aripiprazole is not associated with induction of mania (and in fact may protect against it) and may thus be used safely with patients in whom a bipolar or related disorder has been diagnosed or is suspected.

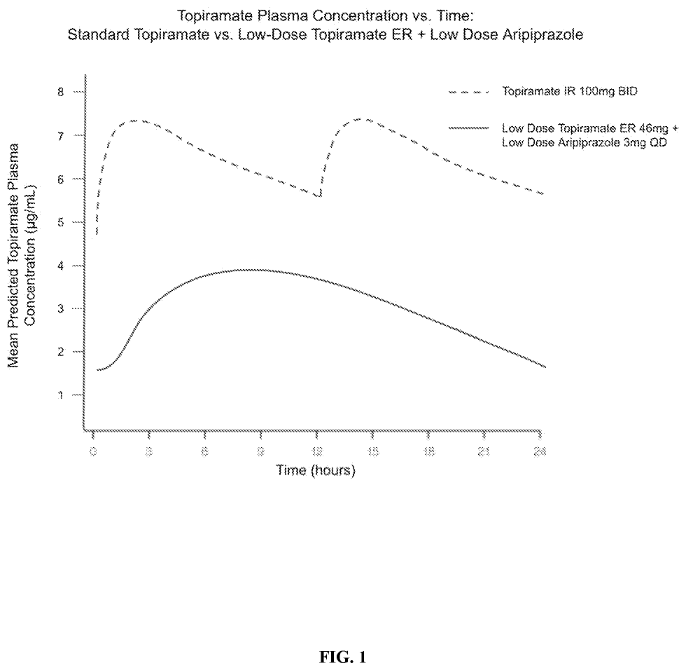

Neither aripiprazole nor topiramate, when administered separately, individually or concurrently to patients, have shown evidence of broad-spectrum efficacy across substance-related and/or addictive disorders and, as such, are not approved for any of these disorders by FDA. Herein described is the discovery that aripiprazole (or a derivative, congener or analogue, e.g., brexpiprazole or cariprazine) and topiramate, when given in combination, or concurrently, are effective in treating individuals suffering from substance-related and/or addictive disorders. In addition, the pharmacodynamic effects of these drugs are, unexpectedly, discovered to be synergistic when used as a treatment for substance-related and/or addictive disorders. This synergistic effect is a critical discovery that allows for the administration of doses significantly below FDA recommended doses of topiramate and aripiprazole, thereby decreasing the incidence of adverse side effects while simultaneously achieving efficacy in disorders that the drugs were not previously known to treat (FIG. 1-5).

Herein an escalating dosing regimen is described for administering aripiprazole in combination with controlled-release topiramate, which has been discovered to be a product that unexpectedly results in an effective and well tolerated non-opioid therapeutic for the treatment of substance-related and/or addictive disorders. The present disclosure is directed to this product and methods of using the product. The disclosure provides a number of important advantages vis-a-vis prior treatments of substance-related and/or addictive disorders therapies, especially as an effective alternative, or adjunct, to the use of opioids to treat addiction, as will be described in detail herein.

### DETAILED DESCRIPTION OF THE DRAWINGS

FIG. 1 depicts the topiramate plasma concentration vs. time curve for immediate release topiramate 100 mg twice daily (BID; within FDA approved dose range for therapy of epilepsy) and low dose topiramate ER 46 mg once daily (QD; the median topiramate dose of the present disclosure). As shown in the graph, the low dose extended-release formulation of topiramate in the present disclosure results in a substantially lower peak plasma concentration and total systemic exposure to topiramate than the standard topiramate dosing and formulation used for epilepsy in FDA approved labeling. Given the documented and well-known, dose-dependent adverse effects associated with topiramate (cognitive impairment, sedation, metabolic acidosis) which often limit its use, the substantially lower topiramate peak plasma concentration and total systemic topiramate exposure associated with the present disclosure greatly improves its risk/benefit profile relative to other standard uses of topiramate. This much lower topiramate dose is permitted in the present disclosure by its co-administration with low dose aripiprazole and the surprising synergistic salience-regulating effects of these two agents.

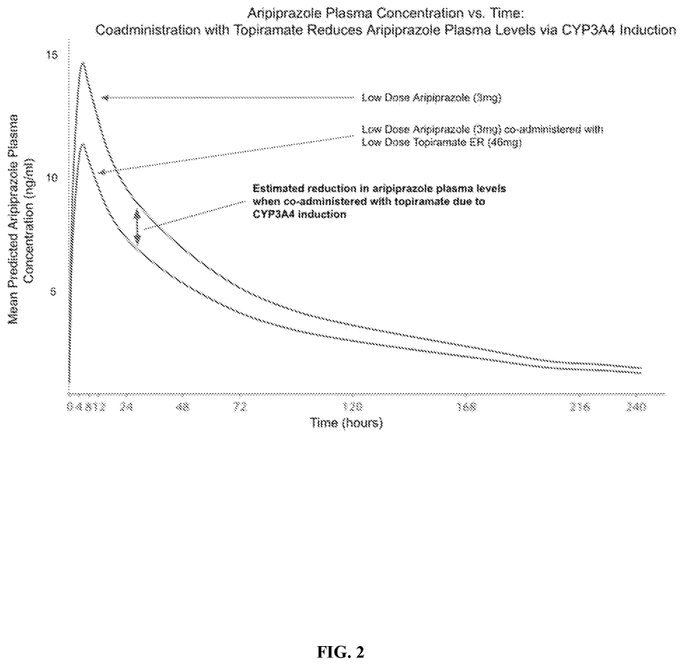

FIG. 2 depicts the predicted aripiprazole plasma concentration vs. time curves for low dose (3 mg) aripiprazole with and without co-administered low dose (46 mg) topiramate ER, as extrapolated from linear PK data. As shown in the graph, there is a predicted 20% reduction in aripiprazole plasma concentration when co-administered with topiramate ER 46 mg due to the latter's induction of CYP3A4.

As noted in the manufacturer's labeling for Abilify (aripiprazole), aripiprazole is metabolized primarily by CYP3A4 and CYP2D6 enzymes and agents that induce CYP3A4 may cause an increase in aripiprazole clearance and lower plasma levels. Topiramate is a known inducer of CYP3A4 and its co-administration has been associated with clinically significant decreases in plasma levels of drugs metabolized by CYP3A4 (Jacob K, Trainer P J. Topiramate can induce hypoadrenalism in patients taking oral corticosteroid replacement, BMJ. 2009 Jan. 8; 338:a1788, doi: 10.1136/bmj.39588.561840.BD. PMID: 19131388.). Co-administration of strong inducers of CYP3A4 (e.g., carbamazepine and phenytoin) has been shown to result in an approximately 60% reduction in steady state plasma levels of aripiprazole (Waade R B, Christensen H, Rudberg I, Refsum H, Hermann M. Influence of comedication on serum concentrations of aripiprazole and dehydroaripiprazole. Ther Drug Monit. 2009 April; 31(2):233-8. doi: 10.1097/FTD.0b013e3181956726. PMID: 19142178). Topiramate may be expected to decrease plasma levels of co-administered aripiprazole to a lesser degree than strong CYP3A4 inducers and is here estimated to do so by approximately 20% (weak inducers of cytochrome P450 enzymes are defined by the FDA to reduce the AUC of sensitive substrates by 20% to 50%; “Drug Development and Drug Interactions, Table of Substrates, Inhibitors and Inducers accessed from the FDA website). The actual reduction in aripiprazole plasma concentration will vary upon patient-specific genetic variations in CYP3A4 activity.

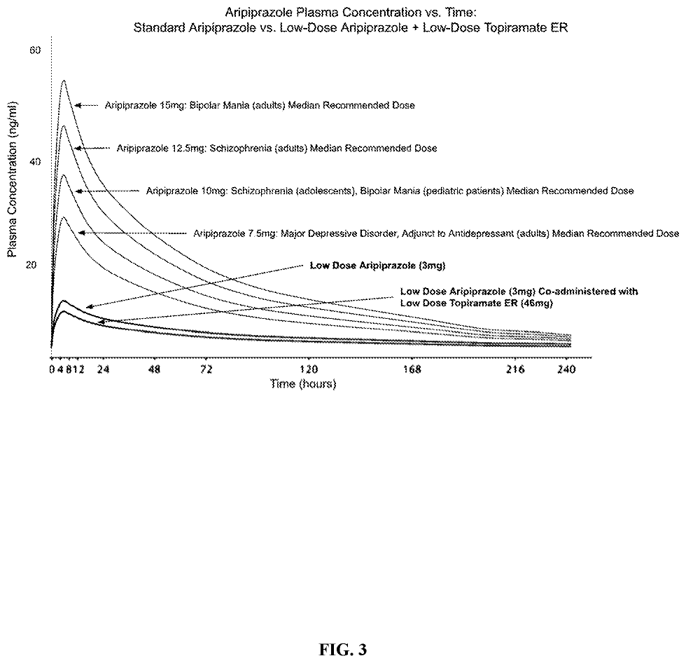

FIG. 3 depicts the predicted aripiprazole plasma concentration vs. time curves for the median dose of aripiprazole for various FDA indications as well as the curves for low dose (3 mg) aripiprazole with and without co-administered low dose (46 mg) topiramate ER. As shown, the present disclosure of co-administered low dose aripiprazole and low dose topiramate ER results in a substantial reduction in both aripiprazole peak plasma concentrations and total systemic aripiprazole exposure (AUC) compared to all current FDA approved uses of aripiprazole. Given the documented and well-known, dose-dependent adverse effects associated with aripiprazole (extrapyramidal symptoms, akathisia [severe restlessness], nausea, weight gain) which often limit its use, the substantially lower aripiprazole peak plasma concentration and total systemic aripiprazole exposure associated with the present disclosure greatly improves its risk/benefit profile relative to other standard uses of aripiprazole. This much lower aripiprazole peak plasma concentration and total systemic aripiprazole exposure is permitted in the present disclosure because of the unexpected discovery that low doses of aripiprazole in combination with low doses of topiramate are effective in the treatment of Substance-related and/or addictive disorders and Substance/Medication-Induced Mental Disorders. Efficacy at doses and plasma levels of these compounds which are currently considered non-therapeutic is a result of their regulation of salience attribution via differing and synergistic mechanisms of action in the mesolimbic pathway in the brain. In addition to the aripiprazole dose reduction permitted by the synergistic salience-regulating effects of co-administered topiramate, the present disclosure achieves further reduction in aripiprazole plasma concentrations by virtue of topiramate's induction of CYP3A4, one of two enzymes primarily responsible for aripiprazole clearance.

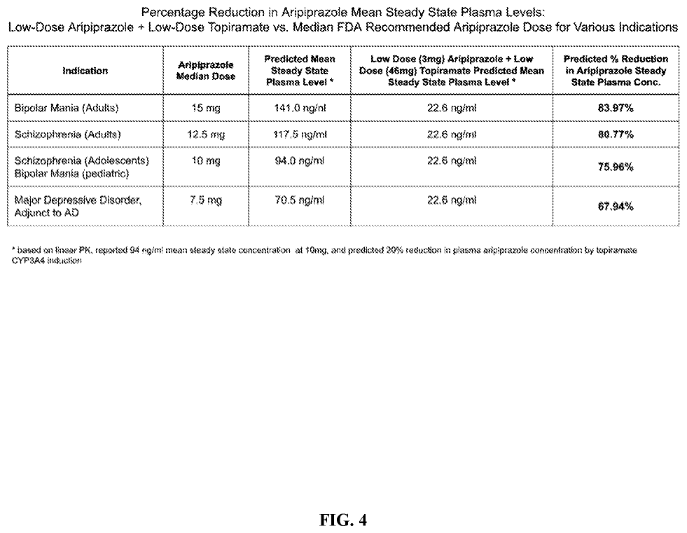

The table in FIG. 4 displays the predicted percentage reduction in aripiprazole steady state plasma levels achieved by the present disclosure (co-administered low dose aripiprazole and low dose topiramate ER), relative to the steady state plasma levels associated with the median aripiprazole doses for other FDA-approved indications for aripiprazole.

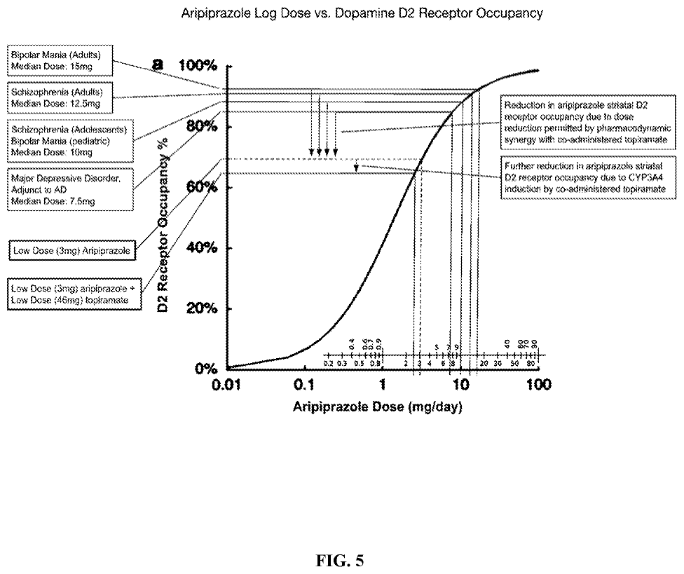

FIG. 5 depicts the aripiprazole striatal dopamine D2 receptor occupancy percentage associated with the median doses of aripiprazole for various FDA indications as well as for low dose (3 mg) aripiprazole with and without co-administered low dose (46 mg) topiramate ER predicted by the log dose vs. striatal D2 receptor occupancy percentage best fit curve from Kegeles et. al. (Kegeles L S, Slifstein M, Frankie W G, Xu X Hackett E, Bae S A, Gonzales R, Kim J H, Alvarez B, Gil R, Laruelle Abi-Dargham A. Dose-occupancy study of striatal and extrastriatal dopamine D2 receptors by aripiprazole in schizophrenia with PET and [18F]fallypride. Neuropsychopharmacology. 2008 December; 33(13):3111-25. doi: 10.1038/npp.2008.33. Epub 2008 Apr. 16. PMID: 18418366) As shown in the graph, low dose (3 mg) aripiprazole is associated with substantially reduced striatal dopamine D2 receptor occupancy relative to the median recommended doses for current FDA approved indications due to this low dose being within the exponential region of the log dose vs. striatal D2 receptor occupancy percentage curve. Also depicted is the further reduction in striatal D2 receptor occupancy achieved in the present disclosure via topiramate's induction of CYP3A4 which increases clearance of aripiprazole and thus reduces aripiprazole plasma levels, here estimated to be a 20% reduction in aripiprazole plasma levels. There is a linear relationship between aripiprazole dose and plasma concentration (Kegeles et. al. 2008) thus the predicted 20% reduction in aripiprazole plasma concentration due to CYP3A4 induction by co-administered topiramate is represented on the log dose vs. D2 receptor occupancy percentage graph by an aripiprazole dose of 2.4 mg (a 20% reduction in the aripiprazole dose of 3 mg).

Importantly, through the dual actions of aripiprazole dose reduction (permitted by synergistic effects with co-administered topiramate) and increased aripiprazole clearance (through CYP3A4 induction by co-administered topiramate) the present disclosure reduces aripiprazole plasma concentrations well into the exponential region of the log dose vs. D2 receptor occupancy percentage curve and thus reduces D2 receptor occupancy and associated adverse effects to a much larger degree than would equivalent percentage reductions in aripiprazole dose in the higher aripiprazole dosing range used for current FDA indications. The common adverse effects of aripiprazole (extrapyramidal symptoms, akathisia, nausea, weight gain) are mediated by excessive D2 blockade and off-target effects at other receptor sites. Potentiation of the salience-regulating effects of aripiprazole by co-administration with topiramate permits efficacy for the treatment of Substance-related and/or addictive disorders and Medication/Substance-Induced Mental Disorders at low doses resulting in lower dopamine D2 receptor occupancy and fewer adverse effects from excessive D2 blockade and off target actions at other receptor sites. Thus, in the present disclosure, topiramate confers both pharmacodynamic and pharmacokinetic benefits when co-administered with aripiprazole for the purposes of treating Substance-related and/or addictive disorders and Medication/Substance-Induced Mental Disorders.

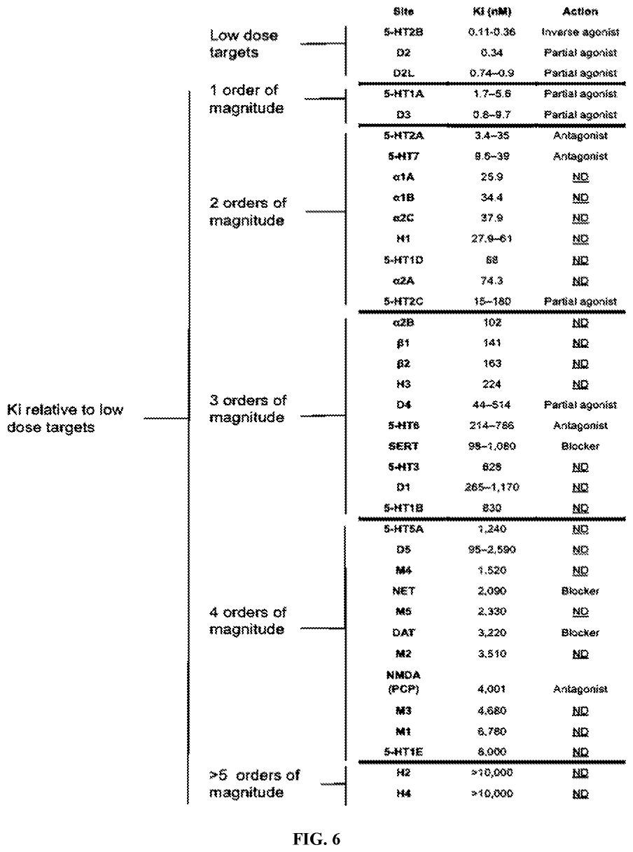

FIG. 6 lists the various binding sites of aripiprazole ordered from higher to lower affinity with associated Ki and action. As shown, aripiprazole is a highly non-selective ligand with interactions on at least 35 CNS receptor sites. This broad range of action contributes to the numerous adverse effects associated with aripiprazole when used at the recommended doses for current FDA indications. Notably, the affinity for these various binding sites, as shown in FIG. 2, spans over 4 orders of magnitude. Thus, by utilizing the surprising synergistic effects of co-administered topiramate and aripiprazole when used for Substance-related and/or addictive disorders and Medication/Substance-Induced Mental Disorders, the present disclosure limits aripiprazole plasma levels to a range in which only it's high-affinity binding sites are appreciably bound and thereby substantially reduces or eliminates the adverse effects mediated by aripiprazole's action at its numerous lower-affinity binding sites. Importantly, the selectivity of lower doses of aripiprazole is for the dopamine and serotonin receptor subtypes critical for its therapeutic effect in the treatment of Substance-related and/or addictive disorders and Medication/Substance-Induced Mental Disorders. Thus, with respect to binding profile, CNS effects and efficacy in the treatment of Substance-related and/or addictive disorders and Medication/Substance-Induced Mental Disorders, aripiprazole administered within the low dosing range permitted in the present disclosure by synergistic action with low dose topiramate, is effectively a different and substantially more selective, safe and efficacious agent than aripiprazole administered alone at the higher doses recommended for current FDA approved indications.

### Definitions and Nomenclature

While the terms used herein are believed to be well understood by one of ordinary skill in the art, definitions are set forth herein to facilitate explanation of the subject matter disclosed herein.

Unless defined otherwise, all technical and scientific terms used herein have the same meaning as commonly understood by one of ordinary skill in the art to which the subject matter disclosed herein belongs. Although any methods, devices, and materials similar or equivalent to those described herein can be used in the practice or testing of the presently disclosed subject matter, representative methods, devices, and materials are described herein.

The terms “a”, “an”, and “the” refer to “one or more” when used in this disclosure, including the claims. The use of the word “a” or “an” when used in conjunction with the term “comprising” in the claims and/or the specification may mean “one,” but it is also consistent with the meaning of “one or more,” “at least one,” and “one or more than one.”

All references to singular characteristics or limitations of the present disclosure shall include the corresponding plural characteristic(s) or limitation(s) and vice versa, unless otherwise specified or clearly implied to the contrary by the context in which the reference is made.

The methods and formulations of the present disclosure, including components thereof, can comprise, consist of, or consist essentially of the essential elements and limitations of the embodiments described herein, as well as any additional or optional components or limitations described herein or otherwise useful.

Unless otherwise indicated, all numbers expressing physical dimensions, quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed. The term “about” includes the values for a number or range disclosed herein that would be known and understood by one of ordinary skill in the art.

When referring to an active agent, applicants intend the term “active agent” to encompass not only the specified molecular entity but also its pharmaceutically acceptable, pharmacologically active analogs, including, but not limited to, salts, esters, amides, prodrugs, conjugates, active metabolites, and other such derivatives, congeners, analogs, and related compounds as will be discussed infra. It is to be understood that when amounts or doses of an active agent are specified, that those amounts or doses refer to the amount or dose of the active agent per se and not to an active agent salt or the like.

The terms “treating”, and “treatment” as used herein refer to reduction in severity and/or frequency of the symptoms or disorder, elimination of the symptoms or disorder, or elimination of the underlying cause of the disorder itself. In certain aspects, the term “treating” and “treatment” as used herein refer to the prevention of the occurrence of symptoms or the disorder, and may be synonymous with “prophylaxis” for the symptoms or disorder. The phrase “administering to a subject” refers to the process of introducing a pharmaceutical composition or dosage form of the disclosure into the subject (e.g., a human or other mammalian subject) via an art-recognized means of introduction.

As used herein, “subject” or “individual” or “patient” refers to any subject for whom or which therapy is desired, and generally refers to the recipient of the therapy to be practiced according to the disclosure. The subject can be any vertebrate but will typically be a mammal. If a mammal, the subject will in many embodiments be a human, but may also be a domestic livestock, laboratory subject or pet animal.

By the terms “effective amount” and “therapeutically effective amount” of an agent, compound, or drug, denote a composition or combination of the disclosure which is nontoxic and effective for producing some desired therapeutic effect upon administration to a subject or patient (e.g., a human subject or patient).

The term “dosage form” denotes any form of a pharmaceutical composition that contains an amount of active agent sufficient to achieve a therapeutic effect with a single administration. When the formulation is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics, such as hydrophilicity.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or “active”) as in a “pharmacologically active” (or “active”) derivative or analog, refers to for example a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, “controlled release” denotes release of an active pharmaceutical agent that is not an immediate release. In some embodiments, a controlled release is an extended release. In other embodiments, a controlled release is a delayed release. As used herein, “controlled release” and “modified release” are used interchangeably.

The terms “controlled release” and “modified release” encompass “extended release” and “delayed release” formulations, as well as formulations having both extended-release and delayed-release characteristics. An “extended-release” formulation can extend the period over which drug is released or targeted to the desired site. A “delayed-release” formulation can be designed to delay the release of the pharmaceutically active compound for a specified period. Such formulations are referred to herein as “delayed-release” or “delayed-onset” formulations or dosage forms. Modified-release formulations of the present disclosure include those that exhibit both a delayed- and extended-release, e.g., formulations that only begin releasing after a fixed period of time or after a physicochemical change has occurred, for example, then continue releasing over an extended period. As used herein, the term “immediate release formulation,” is meant to describe those formulations in which more than about 50% of active ingredient is released from the dosage form in less than about 2 hours.

In general, extended-release drug products are dosage forms of the pharmaceutical composition that allow at least a two-fold reduction in dosage frequency as compared to that drug presented as an immediate-release (conventional) dosage form. Examples of extended-release dosage forms include controlled-release, sustained-release, and long-acting drug products.

In general, delayed-release drug products are dosage forms that release a discrete portion or portions of drug at a time other than promptly after administration. An initial portion may be released promptly after administration. Enteric-coated dosage forms are common delayed-release products (e.g., enteric-coated aspirin and other NSAID products).

### Conditions/Indications

Disclosed herein are methods and compositions for treating Substance Use Disorders, including, but not limited to, Opioid Use Disorder, Cannabis Use Disorder, Tobacco Use Disorder, Sedative, Hypnotic, or Anxiolytic Use disorder, Stimulant Use Disorder, and Alcohol Use Disorder, as well as for treating the recognized Non-Substance-Related Disorder known as Gambling Disorder. Additionally, disclosed herein are methods and compositions for treating Substance/Medication Induced Mental Disorders encompassing the Substance/Medication-Induced Depressive Disorders, Bipolar and Related Disorders, and Anxiety Disorders (defined in DSM-5 within Depressive Disorders, Bipolar and Related Disorders, and Anxiety Disorders) including, but not limited to, Opioid-Induced Depressive Disorder, Amphetamine (or other stimulant)-Induced Depressive Disorder, Cocaine-Induced Depressive Disorder, Sedative-, Hypnotic-, or Anxiolytic-Induced Depressive Disorder, and Alcohol-Induced Depressive Disorder, as well as Opioid-Induced Anxiety Disorder, Cannabis-Induced Anxiety Disorder, Amphetamine (or other stimulant)-Induced Anxiety Disorder, Cocaine-Induced Anxiety Disorder, Sedative-, Hypnotic-, or Anxiolytic-Induced Anxiety Disorder, and Alcohol-Induced Anxiety Disorder, Opioid-Induced Bipolar and Related Disorder, Cannabis-Induced Bipolar and Related Disorder, Amphetamine (or other stimulant)-Induced Bipolar and Related Disorder, Cocaine-Induced Bipolar and Related Disorder, Sedative-, Hypnotic-, or Anxiolytic-Induced Bipolar and Related Disorder, and Alcohol-Induced Bipolar and Related Disorder.

The Diagnostic and Statistical Manual of Mental Disorders, 5th Edition (“DSM-5”) provides diagnostic criteria for substance use disorder, including a cluster of cognitive, behavioral, and physiological symptoms indicating that the individual continues using the substance despite significant substance-related problems. An important characteristic of substance use disorders is an underlying change in brain circuits that may persist beyond detoxification, particularly in individuals with severe disorders. The behavioral effects of these brain changes may be exhibited in the repeated relapses and intense drug cravings when the individuals are exposed to drug related stimuli. These persistent drug effects may benefit from long term approaches to treatment.

The diagnosis of a substance use disorder is based on a pathological pattern of behaviors related to use of the substance. These include criteria that include impaired control, cravings, social impairment, risky use of the substance and the pharmacological criteria of tolerance and withdrawal. In particular, it is known that opioids produce high levels of positive reinforcement, increasing the likelihood that an individual will continue using them despite negative consequences. Opioid use disorder is a chronic lifelong disorder, associated with significant morbidity and mortality.

The DSM-5 describes opioid use disorder as a problematic pattern of opioid use leading to problems or distress, with at least two of the following occurring within a 12-month period: (1) taking larger amounts or taking opioids over a longer period than intended; (2) persistent desire or unsuccessful efforts to cut down or control opioid use; (3) spending a great deal of time obtaining or using the opioid or recovering from its effects; (4) craving, or a strong desire or urge to use opioids; (5) problems fulfilling obligations at work, school or home; (6) continued opioid use despite having recurring social or interpersonal problems; (7) giving up or reducing activities because of opioid use; (8) using opioids in physically hazardous situations; (9) continued opioid use despite ongoing physical or psychological problems likely to have been caused or worsened by opioids; (10) tolerance (i.e., a need for increased amounts or diminished effect with continued use of the same amount); and/or (11) experiencing withdrawal (opioid withdrawal syndrome) or taking opioids or a closely related substance to relieve or avoid withdrawal symptoms.

Cannabis use disorder is a highly prevalent substance use disorder, particularly in youth and young adults, reflecting its widespread use. According to the DSM-5, cannabis use disorder is defined by the use of cannabis for at least a one-year period, with the presence of at least two of the following symptoms, accompanied by significant impairment of functioning and distress: (1) difficulty containing use of cannabis (i.e., the drug is used in larger amounts and over a longer period than intended); (2) repeated failed efforts to discontinue or reduce the amount of cannabis that is used; (3) an inordinate amount of time is occupied acquiring, using, or recovering from the effects of cannabis; (4) cravings or desires to use cannabis (e.g., intrusive thoughts and images, and dreams about cannabis, or olfactory perceptions of the smell of cannabis, due to preoccupation with cannabis); (5) continued use of cannabis despite adverse consequences from its use, such as criminal charges, ultimatums of abandonment from spouse/partner/friends, and poor productivity; (6) other important activities in life, such as work, school, hygiene, and responsibility to family and friends are superseded by the desire to use cannabis; (7) cannabis is used in contexts that are potentially dangerous, such as operating a motor vehicle; (8) use of cannabis continues despite awareness of physical or psychological problems attributed to use (e.g., anergia, amotivation, chronic cough); (9) tolerance to cannabis, as defined by progressively larger amounts of cannabis are needed to obtain the psychoactive effect experienced when use first commenced, or, noticeably reduced effect of use of the same amount of cannabis; and/or (10) withdrawal, defined as the typical withdrawal syndrome associated with cannabis, or cannabis, or a similar substance, is used to prevent withdrawal symptoms.

Tobacco use disorder is currently the most prevalent substance use disorder and cigarette smoking remains the leading preventable cause of disease, disability, and death within the United States, as shown for example by the following CDC statistics). There is also a significant correlation between anxiety disorders and tobacco use disorder with the prevalence of tobacco use among individuals with a severe anxiety disorder nearly three times greater than within the general population (34.5% vs. 12%) (Cornelius M E, Wang T W, Jamal A, Loretan C, Neff L. Tobacco Product Use Among Adults—United States, 2019. Morbidity and Mortality Weekly Report, 2020. Volume 69 (issue 46); pages 1736-1742). According to the DSM-5, tobacco use disorder is defined as a problematic pattern of tobacco use leading to clinically significant impairment or distress, as manifested by at least two of the following symptoms, occurring within a 12-month period: (1) Tobacco is often taken in larger amounts or over a longer period than was intended; (2) there is a persistent desire or unsuccessful efforts to cut down or control tobacco use; (3) a great deal of time is spent in activities necessary to obtain or use tobacco; (4) craving, or a strong desire or urge to use tobacco; (5) recurrent tobacco use resulting in a failure to fulfill major role obligations at work, school, or home; (6) continued tobacco use despite having persistent or recurrent social or interpersonal problems caused or exacerbated by the effects of tobacco (e.g., arguments with others about tobacco use); (7) important social, occupational, or recreational activities are given up or reduced because of tobacco use; (8) recurrent tobacco use in situations in which it is physically hazardous (e.g., smoking in bed); (9) tobacco use is continued despite knowledge of having a persistent or recurrent physical or psychological problem that is likely to have been caused or exacerbated by tobacco; (10) tolerance, as defined by either of the following: (a) A need for markedly increased amounts of tobacco to achieve the desired effect; or (b) a markedly diminished effect with continued use of the same amount of tobacco; and (11) withdrawal, as manifested by either of the following: (a) The characteristic withdrawal syndrome for tobacco (refer to Criteria A and B of the criteria set for tobacco withdrawal); or (b) tobacco (or a closely related substance, such as nicotine) is taken to relieve or avoid withdrawal symptoms.

The DSM-5 also provides diagnostic criteria for substance/medication-induced depressive disorder, which includes a prominent and persistent disturbance in mood that predominates in the clinical picture and is characterized by depressed mood or markedly diminished interest or pleasure in all, or almost all, activities. DSM-5 defined symptoms of a major depressive episode that can be caused by the effects of a psychoactive substance or medication include (1) depressed mood (sadness, emptiness, hopelessness, tearfulness) most of the day, nearly every day; (2) markedly diminished interest or pleasure in all, or almost all, activities; (3) weight loss; (4) insomnia or hypersomnia; (5) agitation or retardation; (6) fatigue or loss of energy; (7) feelings of worthlessness, or excessive or inappropriate guilt; (8) diminished ability to think or concentrate; and/or (9) thoughts of death, recurrent suicidal ideation, or a suicide attempt or plan for committing suicide. These symptoms may occur while the patient is under the influence of the drug (i.e., intoxication) or after use of the drug has stopped (i.e., withdrawal). Symptoms of depression may be precipitated by substance use or withdrawal.

Depression can present in adolescent substance use disorder patients along with symptoms of anergia, amotivation, short-term memory deficits, and irritability, although some of these symptoms may be attributed to substance use and denial, particularly if the adolescent cannot articulate their feelings. To rule out other disorders, in addition to meeting the diagnostic criteria for a substance use disorder, diagnosis of use can be determined through enzyme immunoassay testing of a urine sample for substance metabolites. The presence of metabolites can indicate recent use, and for many substances if quantitative testing is done, the levels of metabolites can be measured, indicating the relative amount of substance(s) recently used.

Similar DSM-5 diagnostic criteria exist for substance/medication-induced anxiety disorder, which is characterized by anxiety or fear, sometimes accompanied by such physiological symptoms as tachycardia (racing heart), dyspnea (breathlessness) and tremor (shakiness), caused by the effects of a medication or psychoactive substance.

These symptoms may occur while the patient is under the influence of the drug (i.e., intoxication) or after use of the drug has stopped (i.e., withdrawal). Generalized anxiety, panic attacks or manifestations of phobia may be precipitated by substance use or withdrawal. Prolonged psychiatric symptoms, including anxiety and panic, can continue for up to six months, and have in some cases been reported for years after cessation of, for example, alcohol, benzodiazepine, and/or opioid use.

Opioids, alcohol, amphetamine and its derivatives, cannabis, cocaine, sedatives, hypnotics, and anxiolytics, hallucinogens, inhalants, and phencyclidine (PCP) have been reported to cause the symptoms of depression or anxiety during, or shortly after, intoxication. Withdrawal from alcohol, cocaine, opioids, cannabis, sedatives, hypnotics, and anxiolytics, amphetamine and its derivatives, and nicotine, can also cause manifestations of depression and anxiety.

Alcohol, cocaine, amphetamine-type substances, sedatives, hypnotics, and anxiolytics may induce and/or exacerbate the symptoms of bipolar and related disorders during, or shortly after, intoxication as well as during withdrawal. Phencyclidine (PCP), hallucinogens, and cannabis may induce and/or exacerbate the symptoms of bipolar and related disorders during, or shortly after, intoxication.

DSM-5 diagnostic criteria for gambling disorder, a non-substance-related disorder, refer to persistent and recurrent problematic gambling behavior leading to clinically significant impairment or distress, and include: (1) A need to gamble with increasing amounts of money in order to achieve the desired excitement; (2) restlessness or irritability when attempting to cut down or stop gambling; (3) repeated unsuccessful efforts to control, cut back, or stop gambling; (4) a preoccupation with gambling (e.g., having persistent thoughts of reliving past gambling experiences, handicapping or planning the next venture, thinking of ways to get money with which to gamble); (5) often gambling when feeling distressed (e.g., helpless, guilty, anxious, depressed); (6) after losing money gambling, often returns another day to get even (“chasing” one's losses); (7) lying to conceal the extent of involvement with gambling; (8) jeopardizes or loses a significant relationship, job, or educational or career opportunity because of gambling; and/or (9) relying on others to provide money to relieve desperate financial situations caused by gambling.

### Methods, Compositions and Formulations of the Disclosure

Clinically diagnosed anxiety, depressive, and bipolar and related disorders very commonly co-occur with substance-related and/or addictive disorders. It is known that there is mutual reinforcement between the symptoms of addictive disorders and those of depressive, bipolar and related, and anxiety disorders when they co-occur. Anxiety/depression and addictive cravings have been observed to be mutually causal. Increased intensity of anxiety and/or depressive symptoms has been shown to be predictive of increased cravings and vice-versa. (Wolitzky-Taylor K, Schiffman J. Predictive Associations Among the Repeated Measurements of Anxiety, Depression, and Craving in a Dual Diagnosis Program. J Dual Diagn. 2019 July-September; 15(3):140-146.) Thus, there exists a need for compositions and methods of treatment which simultaneously target the symptoms of craving, depression and anxiety as well as the mechanisms underlying their mutual reinforcement.

Administration of compositions described herein results in better outcomes in the treatment of individuals suffering from substance related and addictive disorders such as opioid use and/or cannabis use disorder patients. Patients to whom compositions described herein are administered exhibit or demonstrate improvement in substance use disorder symptoms including a reduction in relapse, a reduction in cravings, increased retention in treatment, as well as an improvement in mood and anxiety symptoms, and overall improved functionality.

Moreover, administration of compositions described herein results in better outcomes in the treatment of substance-induced mental disorders, including opioid-induced, cannabis-induced, alcohol-induced, and/or sedative-, hypnotic- or anxiolytic-induced depressive, bipolar, or anxiety disorders. Patients to whom compositions described herein are administered exhibit or demonstrate an improvement in mood demonstrated by a decrease in symptoms of depression, an improvement in anxiety related symptoms, and overall improved functionality.

It is believed that compositions described herein which contain aripiprazole (or a derivative, congener or analogue, e.g., brexpiprazole or cariprazine) and topiramate mediate such improvements via a unique effect on the dopaminergic, serotonergic, GABAergic, and glutamatergic pathways and systems involved in mood and reward. However, neither aripiprazole nor topiramate, when administered separately, individually and not concurrently to patients, have shown evidence of broad-spectrum efficacy across substance related and addictive disorders and, as such, are not approved for any of these disorders by FDA. Herein described is the discovery that aripiprazole and topiramate, when given in combination, or concurrently, are effective in treating individuals suffering from substance related and addictive disorders, including those suffering from substance/medication-induced depressive, bipolar and related, and anxiety disorders.

It is believed that through their direct action on the mesolimbic pathway and other limbic pathways, compositions to be described herein which contain both aripiprazole (or a congener or analogue, e.g., brexpiprazole or cariprazine) and topiramate decrease cravings for substances (and for activities, such as those occurring in gambling disorders, a non-substance related addictive disorder), and attenuate symptoms of substance withdrawal. Their mechanism of action also, simultaneously, alleviates or otherwise improves the mood- and anxiety-related symptoms associated with substance use disorders, including opioid use disorder and cannabis use disorder. These compositions also target the mood and anxiety symptoms and cravings associated with substance-induced mental disorders, such as opioid-induced depressive and/or anxiety disorders. Thus, administration of compositions described herein containing aripiprazole combined with topiramate improve treatment/relapse outcomes in patients with substance-related and/or addictive disorders including those who present with comorbid mood and/or anxiety symptoms which may be nearly all individuals having one or more substance-related or addictive disorders.

The mesolimbic pathway mediates the attribution of salience, or “attention worthiness”, to stimuli and cognitions previously associated with euphoria or distress. In non-pathological states, this function of the mesolimbic pathway promotes survival by helping to motivate the repetition of rewarding behaviors and the avoidance of dangerous or distressing events. In the pathological states of substance-related and/or addictive disorders, anxiety disorders, and depressive disorders, dysregulation of the mesolimbic pathway results in excessive salience assigned to rewarding (substance-related and/or addictive disorders) or aversive (anxiety and depressive disorders) stimuli and cognitions leading to the dysfunctional behaviors and suffering associated with these conditions.

Via different mechanisms, aripiprazole and topiramate modulate neurotransmission in the mesolimbic pathway, and may, therefore, when used in combination, regulate salience attribution and attenuate the symptoms of combined substance-related and/or addictive disorders, depressive disorders, bipolar and related disorders, and anxiety disorders. Unfortunately, both aripiprazole and topiramate are associated with adverse effects that may be particularly problematic in patients with substance use and addictive disorders and could substantially limit achievement of a maximally effective, well-tolerated dose for these conditions. For example, the most common adverse effect associated with aripiprazole is akathisia (severe restlessness), which is uncomfortable for any patient, but is often intolerable to patients in acute or post-acute withdrawal from opioids, alcohol, sedatives, cannabis, or anxiolytics. Similarly, two common adverse effects of topiramate are sedation and a significant cognitive impairment associated with word finding difficulty that limits its use in these patient populations.

Disclosed herein is a combination therapy for treating substance-related and/or addictive disorders, substance induced anxiety disorders, substance induced bipolar disorders, and substance induced depressive disorders, wherein a newly discovered synergistic effect between aripiprazole and topiramate enables achievement of desired efficacy at doses (and associated plasma drug levels) substantially below those of currently labeled recommended doses for other FDA indications, resulting in a concomitant reduction in the adverse effects reported with each active agent in their approved labeling (FIG. 1-5). Thus, the combination treatment discovered will deliver the first well tolerated, effective, non-opioid treatment to a broad spectrum of substance-related and/or addictive disorders.

Importantly, because both aripiprazole (or a derivative, congener or analogue, e.g. brexpiprazole or cariprazine) and topiramate are non-opioids, they can be safely used concomitantly with opioids. Thus, they can be co-administered with opioid-based medications for opioid use disorder when additional efficacy for the substance use disorder is necessary, eliminating the risk of the adverse effects associated with increasing the doses or prolonging the use of opioid therapies (e.g., social and/or emotional dysfunctions, increasing tolerance, endocrine abnormalities). Furthermore, because all current FDA approved medications for opioid use disorder are themselves opioid receptor ligands, none may be co-administered with any of the others or used in patients who are actively using opioids, substantially limiting pharmacologic treatment of opioid use disorder. Conversely, as non-opioids, aripiprazole and topiramate may be used concomitantly with any of the current FDA approved medications for opioid use disorder and in patients actively using opioids. In addition, because they can be safely co-administered with opioids, their efficacy can be leveraged by concomitant administration in patients on opioid therapy to specifically decrease, and eventually entirely eliminate, the need for opioid related drugs and the risks and compromised efficacy they entail.

Another unique and critically important new disclosure the combination of aripiprazole (or a derivative, congener or analogue, e.g., brexpiprazole or cariprazine) and topiramate provides is the prophylaxis or prevention of substance-related and/or addictive disorders. The mechanism by which aripiprazole and topiramate treat substance-related and/or addictive disorders is via attenuation of incentive salience through regulation of mesolimbic neurotransmission. Because this mechanism acts at the final common pathway underlying all substance use and addictive disorders, rather than at the upstream pathways through which potentially addictive substances achieve their effect, aripiprazole/topiramate may be co-administered with these substances to prevent the development of a substance use and/or substance-induced disorder, without the risk of deleterious interactions. A particularly important population that could be administered such a prophylactic regimen are patients prescribed opioid analgesics for post-surgical or chronic pain. Currently, the only medications available for opioid use disorder are opioid receptor ligands themselves and are thus contraindicated in the context of concomitant opioid use, making them unusable for opioid use disorder prophylaxis in this setting and unusable in any patient prescribed opioids for analgesia. Additionally, patients using illicit opioids who are unable or unwilling to abstain could be administered aripiprazole/topiramate to prophylactically prevent progression of their use to opioid use disorder or prevent progression from mild opioid use disorder to moderate or severe opioid use disorder. As opioid receptor ligands, the currently available pharmacologic treatments for opioid use disorder are unusable for these patients.

Another unique and critically important new disclosure the combination of aripiprazole (or a derivative, congener or analogue, e.g., brexpiprazole or cariprazine) and topiramate provides is the prophylaxis or prevention of substance-induced anxiety and depression. Depressive disorders, bipolar and related disorders, and anxiety disorders may be induced as a consequence of the action of a substance or medication or as a consequence of its withdrawal. As an example, patients currently using prescribed or illicit opioids, can be safely administered aripiprazole/topiramate concurrently to prevent or treat opioid induced anxiety or depressive disorders. Additionally, aripiprazole/topiramate may be safely used concurrently with naltrexone (e.g., Vivitrol) to treat or prevent the intolerable side effect of naltrexone-induced depression while simultaneously enhancing the likelihood of positive treatment outcomes for opioid use disorder.

Another patient population for which aripiprazole/topiramate may be used prophylactically to prevent the development of a use disorder are patients receiving benzodiazepines for treatment of an anxiety disorder. Patients with anxiety disorders are particularly susceptible to the development of substance use and addictive disorders, and while benzodiazepines are highly effective in the reduction of anxiety symptoms their use also carries significant risk for addiction in these patients. Because aripiprazole/topiramate reduces incentive salience without affecting benzodiazepine's therapeutic mechanism of action, it may be co-administered with this medication class to reduce the risk of benzodiazepine use disorder development or progression without diminishing benzodiazepine's anxiolytic benefit.

Additionally, because aripiprazole/topiramate is non-addictive, patients with an addictive disorder in remission who are judged to be at risk for relapse due to psychosocial stressors or recreational use of other potentially addictive substances or are in need of treatment with potentially addictive medication for other medical conditions, could be administered aripiprazole/topiramate prophylactically to reduce the risk of relapse to active addiction. Aripiprazole/topiramate may be also used in individuals identified as high risk for a substance-related and/or addictive disorder in order to prevent its development.

Of particular importance is the discovery that the pharmacodynamic effects of these drugs are found, unexpectedly, to be synergistic when used as a treatment for substance related and addictive disorders. This synergistic effect allows for the administration of doses significantly below FDA recommended doses of topiramate (100-400 mg/day for migraine and epilepsy) and aripiprazole (5-30 mg/day in depressive and schizophrenic disorders), thereby decreasing the incidence of adverse side effects while simultaneously achieving efficacy in disorders the drugs were not previously known to treat (FIG. 1-5). Thus, in an exemplary embodiment, the topiramate, or its pharmaceutically acceptable salt, is administered at less than 100 mg per day and aripiprazole, or its derivatives/congeners/analogues or pharmaceutically acceptable salt, is administered at less than 5 mg per day, as disclosed herein. It is to be noted that the adverse effects of topiramate and aripiprazole are particularly problematic in these patient populations and could substantially limit achievement of a maximally effective, well-tolerated dose for these conditions when these drugs are administered independently. In summary, a combination therapy for treating substance-related and/or addictive disorders, substance induced anxiety disorders, substance induced bipolar disorders, and substance induced depressive disorders is provided wherein a newly discovered synergistic effect between aripiprazole and topiramate enables achievement of desired efficacy at doses (and associated plasma drug levels) below currently labeled recommended doses, resulting in a concomitant reduction in the adverse effects reported with each active agent in their approved labeling for other indications. Thus, the combination treatment discovered will deliver the first well tolerated, effective, non-opioid treatment to a broad spectrum of substance-related and/or addictive disorders.

Accordingly, the present disclosure is directed to administering together aripiprazole and topiramate in a medication for treatment and/or prophylaxis of a substance related and addictive disorder, or a substance-induced depressive disorder, bipolar and related disorder, or anxiety disorder, and the present disclosure provides novel methods and mechanisms to treat such disorders.

Certain embodiments described herein are directed to pharmaceutical compositions that include a core containing topiramate. In an aspect, dosage forms described herein may be formulated as once-daily, twice-daily, or three or more times daily dosage forms. Any pharmaceutically acceptable dosage form may be administered according to methods described herein, such as bilayer tablets.

In certain embodiments, a portion (e.g., a film or other layer) covers a topiramate-containing core and the portion includes a polymer layer, and the polymer layer includes a controlled release polymer. In an aspect, the controlled release polymer may be any suitable release polymer, such as a delayed release polymer or an extended-release polymer.

Other non-limiting embodiments described herein are directed to bilayer tablets, wherein aripiprazole is granulated with a binder in an immediate release layer and topiramate is granulated with a binder and a rate-controlling polymer in a modified release (e.g., a delayer release or extended release) layer.

As used herein, “aripiprazole” denotes aripiprazole as well as any pharmaceutically acceptable salt or a derivative, congener or analogue (e.g., brexpiprazole or cariprazine) thereof.

As used herein, “topiramate” denotes topiramate as well as any pharmaceutically acceptable salt thereof.

In an aspect, the rate controlling polymer may be any pharmaceutically acceptable delayed release polymer or any pharmaceutically acceptable extended-release polymer.

Non-limiting examples of suitable rate-controlling polymers include methyl acrylate-methacrylic acid copolymers, cellulose acetate phthalate (CAP), cellulose acetate succinate, hypromellose phthalates (e.g., hypromellose phthalate RP-55), hydroxypropyl methyl cellulose phthalates, hydroxypropyl methyl cellulose acetate succinates (hypromellose acetate succinate), talc, polyvinyl acetate phthalates (PVAP), methyl methacrylate-methacrylic acid copolymers, shellac, cellulose acetate trimellitate, sodium alginate, zein, enteric coating aqueous solutions such as ethylcellulose (e.g., ethylcellulose 100 premium), medium chain triglycerides [e.g., coconut], oleic acid, sodium alginate, and/or stearic acid. Suitable rate controlling polymers may also include, without limitation, one or more of the following polymers: EUDRAGIT® NM 30D, ethylcellulose, polyvinylpyrrolidone, polylactides (PLA), polyglycolides (PGA), poly(lactide-co-glycolides) (PLGA), polyanhydrides, and polyorthoesters.

As appropriate, a skilled artisan can select a suitable polymer from hydrophilic polymers, non-cellulosic polymers, and hydrophobic polymers, for example. Hydrophilic polymers include, but are not limited to, methylcellulose, hydroxypropylmethylcellulose (HPMC), hydroxypropyl cellulose (HPC), hydroxyethylcellulose (HEC), ethyl hydroxyethyl cellulose (E-HEC), and sodium-carboxymethylcellulose (Na-CMC), for example. Non-cellulosic polymers include, but are not limited to, sodium alginate, xanthan gum, carrageenan, chitosan, guar gum, pectin, and polyethylene oxide, for example. Hydrophobic polymers include, but are not limited to, ethylcellulose, hypromellose acetate succinate, cellulose acetate, cellulose acetate propionate, for example.

In an aspect, a delayed release polymer described herein may be a pH dependent polymer.

In an aspect, the granulation may be carried out by any known means of granulation. In certain embodiments, an active pharmaceutical ingredient is granulated with a binder by high shear wet granulation. In other embodiments, an active pharmaceutical ingredient is granulated with a binder by fluid bed granulation. In certain embodiments, following granulation, an active pharmaceutical ingredient is mixed with extragranular excipients.

In embodiments comprising modified release drug layers, topiramate is granulated with a binder and rate-controlling polymer by any known means of granulation. In certain embodiments, topiramate is granulated with a binder and rate-controlling polymer by high shear wet granulation. In other embodiments, topiramate is granulated with a binder and rate-controlling polymer by high shear wet granulation.

In an aspect, bilayer tablet formulations described herein may comprise one immediate release layer and one extended-release layer. In another aspect, bilayer tablet formulations described herein may comprise one immediate release layer and one delayed release layer. In yet another aspect, bilayer tablet formulations described herein may comprise one extended-release layer and one delayed release layer. In another aspect, bilayer tablet formulations described herein may comprise the same or different release layers that may be immediate release, delayed release or extended release.

In certain embodiments, to prepare an immediate release drug layer described herein, aripiprazole is granulated with at least one binder. In such embodiments, a modified release drug layer described herein may be prepared by granulating topiramate with at least one binder and at least one rate-controlling polymer.

In an aspect, any suitable, pharmaceutically acceptable binder may be used. In some embodiments the binder is one or more starches, microcrystalline cellulose, hydroxypropyl cellulose, hydroxypropylmethylcellulose, hydroxyethyl cellulose, hydroxypropylmethyl cellulose, a polyvinyl alcohol and/or polyvinylpyrrolidone.

In an aspect, following granulation, an active pharmaceutical ingredient is mixed with one or more extragranular excipients.

Examples of suitable excipients for use in the compositions or dosage forms described herein may include fillers, diluents, glidants, disintegrants, binders, lubricants etc. Other pharmaceutically acceptable excipients may include acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents, etc. In an aspect, any pharmaceutically acceptable excipients may be included.

In an aspect, any suitable core structure may be used. The drug core may have one, two, three, or more drug layers with one, two, three or more protective layers around each drug layer. In certain embodiments, the core is an immediate release core wherein the core is a compressed core surrounded by at least an immediate release coating or film. In other embodiments, the core is an extended-release core.

In still other embodiments, the plural tablets include a gel matrix extended-release core. In the case of a gel matrix core, such as a hydrophilic matrix system, a covalently crosslinked gel matrix may be used, for example. In a gel matrix core structure, the undissolved active pharmaceutical ingredient, e.g., topiramate, is contained in a dry polymer zone surrounded by a gel layer zone in which active ingredients may be dissolved and dispersed. Additional gel layers may be included, optionally containing dissolved active ingredient molecules. The primary mechanism of drug release from hydrophilic matrices occurs when the polymer swells on contact with the aqueous medium to form a gel layer on the surface of the system. The drug then releases by dissolution, diffusion and/or erosion.

In an aspect, a skilled artisan can select a suitable polymer from those commonly known in the art and discussed herein.

In certain embodiments of the present disclosure, any of the once-daily, twice-daily, or three-times-daily pharmaceutical compositions may include plural tablets wherein each tablet is a bilayer tablet as described herein. In such embodiments, both aripiprazole and topiramate are included as active pharmaceutical ingredients.

In other embodiments of the present disclosure, any of the once-daily, twice-daily, or three-times-daily pharmaceutical compositions may include plural tablets wherein some of the plural tablets contain aripiprazole and other tablets contain topiramate. In certain such embodiments, a first portion encloses the tablets which contain topiramate. The first portion includes a polymer layer.

In certain embodiments, the first portion may be a film portion or layer.

In certain embodiments, the polymer layer includes a controlled release polymer.

In certain embodiments, the controlled release polymer is a delayed release polymer. In other embodiments, the controlled release polymer is an extended-release polymer. In still other embodiments, the plural tablets include a gel matrix extended-release core.

In certain embodiments comprising plural tablets, the plural tablets containing aripiprazole are enclosed by a second portion which includes an extended-release polymer.

In certain embodiments comprising plural tablets, the plural tablets include an immediate release core. A second portion covers the immediate release core, and the second portion includes an extended-release polymer. In an aspect, the plural tablets may be provided in a formulation in any suitable manner for effective pharmaceutical delivery of the active ingredients.

In an exemplary embodiment, a capsule described herein contains at least one immediate release tablet and at least one delayed release tablet. In such embodiments, the immediate release tablet(s) contain(s) aripiprazole as an active pharmaceutical ingredient and the delayed release tablet(s) contain(s) topiramate as an active pharmaceutical ingredient.

In another exemplary embodiment, a capsule described herein contains at least one immediate release tablet and at least one extended-release tablet. In such embodiments, the immediate release tablet(s) contain(s) aripiprazole as an active pharmaceutical ingredient and the extended-release tablet(s) contain(s) topiramate as an active pharmaceutical ingredient.

In an aspect, in a capsule formulation described herein, all the tablets of at least one type are uniform in size and each have a diameter between 3-10 mm or between 5-8 mm.

In another embodiment, the plural tablets further include at least one of the following: a gel matrix extended-release core, or an immediate release core in which a second portion covers the immediate release core, and the second portion includes at least one extended release polymer.

In embodiments containing a gel matrix extended-release core, a covalently crosslinked gel matrix may be used, for example. In a gel matrix core structure, topiramate is the active pharmaceutical ingredient and it remains undissolved. In particular, topiramate is contained in a dry polymer zone surrounded by a gel layer zone in which active ingredients may be dissolved and dispersed. In some embodiments, additional gel layers may be included, optionally containing dissolved active ingredient molecules.

Another aspect of the present disclosure is directed to a pharmaceutical composition that includes plural tablets. The tablets may contain the same or different active ingredients, inactive ingredients, or combinations of the same. A first portion or film encloses the plural tablets. The first portion includes a polymer layer, and the polymer layer may include at least one controlled release polymer.

In one embodiment, the at least one controlled release polymer is a delayed release polymer.

In another embodiment, the at least one controlled release polymer is an extended-release polymer.

In another embodiment, the at least one controlled release polymer includes one or more of the following polymers: EUDRAGIT® NM 30D, ethylcellulose, polyvinylpyrrolidone, polylactides (PLA), polyglycolides (PGA), poly(lactide-co-glycolides) (PLGA), polyanhydrides, and polyorthoesters.

In another embodiment, one or more of the plural tablets includes a gel matrix extended-release core.

In another embodiment, one or more of the plural tablets includes an immediate release core. A second portion covers the immediate release core, and the second portion includes an extended release polymer.

In another embodiment, the extended-release polymer layer includes one or more of the following polymers: EUDRAGIT® NM 30D, ethylcellulose, polyvinylpyrrolidone, polylactides (PLA), polyglycolides (PGA), poly(lactide-co-glycolides) (PLGA), polyanhydrides, and polyorthoesters.

In another embodiment, the plural tablets are stacked on top of one another. This embodiment may take the form of a bilayer tablet containing different release characteristics whether they be delayed release, immediate release or any variation therein for each layer.

Core tablets described herein may be prepared by roller compaction and tablet compression techniques that are well known to one of ordinary skill in the art.

Core tablets are coated with a controlled release polymer layer (e.g., EUDRAGIT® NM or ethylcellulose or other similar polymers or combinations of polymers).

Other polymers or combination of polymers can include but are not limited to poly (2-hydroxyethyl methacrylate), poly (N-vinyl pyrrolidone), poly (methyl methacrylate), poly (vinyl alcohol), poly (acrylic acid), polyacrylamide, poly (ethylene-co-vinyl acetate), poly (ethylene glycol), poly (methacrylic acid), polylactides (PLA), polyglycolides (PGA), poly (lactide-co-glycolides) (PLGA), polyanhydrides, polyorthoesters, EUDRAGIT® L 30 D-55, EUDRAGIT® FS 30 D, EUDRAGIT® L, EUDRAGIT® S, EUDRAGIT® FL 30 D-55, and EUDRAGIT® RL.

In an aspect, a once daily dosage form described herein may comprise administration or ingestion of one, two, three, four, five or more tablets or capsules at once in multiple dosage forms or a single dosage form. For example, one, two or three modified release tablets or capsules may be administered in the morning, depending on the amount of active ingredient contained in each tablet or capsule. In an aspect, a once daily dosage form described herein may be administered to a patient in the form of 1, 2, 3, 4, 5, 6 tablets at a time. In an embodiment, a once daily dosage form is administered to a patient in the form of 1, 2, 3, or 4 tablets at a time. In a further embodiment, a once daily dosage form is administered to a patient in the form of 2 or 3 tablets at a time.

In an aspect, a twice daily dosage form described herein may comprise administration of one, two, three, four, five or more tablets or capsules at once. In an exemplary embodiment for twice daily dosage, a dosage form of one or two tablets is administered at once. For example, one or two tablets or capsules may be administered in the morning and one or two capsules may be administered in the evening.

In an aspect, a three times daily pharmaceutical composition or dosage form described herein may comprise administration of one, two, three, four, five or more tablets or capsules at once. In an embodiment for three times daily dosage, a dosage form of one or two tablets is administered at once. For example, one tablet or capsule may be administered at a time, three different times throughout the day.

In an aspect, a pharmaceutical composition or dosage form described herein may comprise aripiprazole in a total amount of 0.5 to 12 mg per dosage form.

In an aspect, a pharmaceutical composition or dosage form described herein may comprise topiramate in a total amount of 5 to 150 mg per dosage form.

In one aspect the pharmaceutical compositions or dosage forms are 1.5 mg aripiprazole in combination with 23 mg topiramate, 3 mg aripiprazole in combination with 46 mg topiramate, 4.5 mg aripiprazole in combination with 69 mg topiramate and 6 mg aripiprazole in combination with 92 mg topiramate.

In an aspect, according to the pharmaceutical composition or dosage form described herein, aripiprazole and topiramate are administered in a weight ratio aripiprazole:topiramate of between about 1:1 to about 1:50.

In an aspect, a composition described herein comprises a controlled release polymer which is selected such that topiramate is released in a controlled manner such that the concentration of topiramate in the bloodstream provides a steady state level consistent with aripiprazole.

In an aspect, embodiments herein may comprise one or more binders. For example, embodiments described herein may comprise Povidone binders, such as Povidone USP (Kollidon or other commercially available binders. Tablets described herein may comprise one or more binders in an amount of 0.1 to 100 mg/tablet, or preferably 0.5 to 75 mg/tablet, or more preferably 1 to 50 mg/tablet. For example, about 5, 10, 15, 20, 25, 30, 35, 40, 45, 50, 55, 60 or 65 mg.

In an aspect, embodiments herein may comprise one or more wetting agents. For example, embodiments described herein may comprise colloidal silicon dioxide, NF (Aerosil 200), or other commercially available wetting agents. Tablets described herein may comprise one or more superdisintegrants in an amount of 0.1 to 100 mg/tablet, or preferably 0.5 to 75 mg/tablet, or more preferably 1 to 50 mg/tablet. For example, the amount of wetting agent can be based on the weight of the solid formulation. A wetting agent/or dispersing agent is present for example in an amount of about 0.1 mg to 10 mg/tablet.

In an aspect, embodiments herein may comprise one or more lubricants. For example, embodiments described herein may comprise Compritol 888 ATO, or other commercially available lubricants. Such as magnesium stearate. Tablets described herein may comprise one or more lubricants in an amount of 0.1 to 100 mg/tablet, or preferably 0.5 to 75 mg/tablet, or more preferably 1 to 50 mg/tablet. For example, the lubricant is present in an amount of 0.7-1.5% (wt % of total (coated) tablet mass).

In an aspect, embodiments herein may comprise one or more superdisintegrants. For example, embodiments described herein may comprise sodium starch glycolate, or other commercially available wetting agents. Tablets described herein may comprise one or more superdisintegrants in an amount of 0.1 to 100 mg/tablet, or preferably 0.5 to 75 mg/tablet, or more preferably 1 to 50 mg/tablet. For example, in a concentration up to 40% by weight, or 20-40% by weight.

In an aspect, modified release coatings of embodiments described herein preferably comprise one or more rate controlling polymers, such as extended-release polymers or delayed release polymers. For example, embodiments described herein may comprise ethylcellulose 100 premium or other commercially available rate controlling polymers. Tablets described herein may comprise one or more rate controlling polymers in an amount of 0.1 to 100 mg/tablet, or preferably 0.5 to 75 mg/tablet, or more preferably 1 to 50 mg/tablet. For example, the rate controlling polymers are present in an amount of about 3% to about 30% by weight.

In an aspect, extended-release coatings of embodiments described herein preferably comprise one or more pore formers. For example, embodiments described herein may comprise Povidone USP (Plasdone K90) or other commercially available pore formers. Tablets described herein may comprise one or more rate controlling polymers in an amount of 0.1 to 100 mg/tablet, or preferably 0.5 to 75 mg/tablet, or more preferably 1 to 50 mg/tablet. For example, the pore former may be present in an amount ranging from about 20% to 45%, about 25% to 45%, about 30% to 45%, and about 35% to 45% by weight of the functional coating composition. In one such embodiment, the pore former may be present in an amount selected from about 20%, 25%, 30%, 35%, 40%, 45%, and 50% by weight of the functional coating composition.

In an aspect, extended-release coatings of embodiments described herein preferably comprise one or more plasticizers. For example, embodiments described herein may comprise a polyethylene glycol or other commercially available plasticizer. For example, embodiments described herein may comprise PEG 1450 granular or other PEGs. Tablets described herein may comprise one or more plasticizers in an amount of 0.1 to 100 mg/tablet, or preferably 0.5 to 75 mg/tablet, or more preferably 1 to 50 mg/tablet. For example, about 5, 10, 15, 20, 25, 30, 35, 40, 50, 55, 60 or 65 mg.

In an aspect, delayed release coatings of embodiments described herein preferably comprise one or more pH dependent polymers. For example, embodiments described herein may comprise hypromellose phthalate HP-55 or other commercially available pH dependent polymers. Tablets described herein may comprise one or more pH dependent polymers in an amount of 0.1 to 200 mg/tablet, or preferably 0.5 to 175 mg/tablet, or more preferably 1 to 150 mg/tablet. For example, about 5, 10, 15, 20, 25, 30, 35, 40, 45, 50, 55, 60, 65, 70, 75, 80, 85, 90, or 100 mg.

In an aspect, delayed release coatings of embodiments described herein preferably comprise one or more anti-tacking agents. For example, embodiments described herein may comprise talc or other commercially available anti-tacking agents. Tablets described herein may comprise one or more anti-tacking agents in an amount of 0.1 to 100 mg/tablet, or preferably 0.5 to 75 mg/tablet, or more preferably 1 to 50 mg/tablet. For example, about 5, 10, 15, 20, 25, 30, 40, 45, 50, 55, 60 or 65 mg.

In an aspect, tablets described herein may have a total weight of between 250 and 2000 mg or 500 and 1500 mg or 750 and 1000 mg. For example, the tablets may have a total weight of about 250, 500, 1000, 1500, 2000, or about 2500 mg.

Various formulations can be prepared with different release profiles by modifying the controlled release polymer layer composition.

Small core tablets may be prepared by roller compaction and tablet compression techniques that are well known to one of ordinary skill in the art.

Core tablets are coated with a controlled release polymer layer (e.g., EUDRAGIT® NM or ethylcellulose or other similar polymers or combinations of polymers).

Other polymers or combination of polymers can include but are not limited to poly (2-hydroxyethyl methacrylate), poly (N-vinyl pyrrolidone), poly (methyl methacrylate), poly (vinyl alcohol), poly (acrylic acid), polyacrylamide, poly (ethylene-co-vinyl acetate), poly (ethylene glycol), poly (methacrylic acid), polylactides (PLA), polyglycolides (PGA), poly (lactide-co-glycolides) (PLGA), polyanhydrides, polyorthoesters, EUDRAGIT® L 30 D-55, EUDRAGIT® FS 30 D, EUDRAGIT® L, EUDRAGIT® S, EUDRAGIT® FL 30 D-55, and EUDRAGIT® RL.

Various formulations can be prepared with different release profiles by modifying the controlled release polymer layer composition.

In certain embodiments, small tablets are stacked on top of each other and enclosed in a capsule. A benefit of this approach compared to using extended-release beads is that the stacked small tablets can maximize the space in the capsule and leave less free space. The formulation of the small tablets can be, for example, either gel matrix extended release or immediate release cores with an extended-release coating.

In an embodiment, all the tablets in a capsule include a gel matrix extended-release core. In another embodiment, all the tablets in a capsule include an immediate release core. In certain embodiments, a second portion covers the immediate release core, and the second portion includes an extended-release polymer.

In an aspect, the size of the tablets can be from 1 to 7 mm in diameter. Different sizes will allow for placement in different size capsules. In certain embodiments, the capsules are sized at a high tolerance designed to hold the tablets with minimum movement.

In other embodiments, different small tablets in the capsules contain different Active Pharmaceutical Ingredients (APIs) that have distinct release profiles (e.g., extended release for one type of tablet and delayed release for another).

A non-limiting example of plural small tablets contained in a capsule is provided.

In an aspect, pharmaceutically acceptable carriers, excipients, or stabilizers which are nontoxic to the cell or mammal being exposed thereto at the dosages and concentrations employed may also be included the tablets/capsules.

The physiologically acceptable carrier may be a sterile aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants. Examples of the surfactant include sodium lauryl sulfate, polysorbate, polyoxyethylene hydrogenated castor oil and the like.

Also described herein are methods of treating at least one substance use disorder.

In an aspect, one or more compositions described herein may be administered to a patient for treatment or prophylaxis of any substance-related or addictive disorder. In certain embodiments, a single composition may be administered which contains topiramate and aripiprazole (or a derivative, congener or analogue, e.g., brexpiprazole or cariprazine). In other embodiments, multiple compositions may be administered together, each comprising one or more active ingredients.

In certain embodiments, the substance-related and/or addictive disorder is selected from the group consisting of a substance-induced disorder, such as substance intoxication or substance withdrawal, and a substance use disorder (alternatively known as substance abuse or substance dependence). Additionally, or alternatively, the methods described herein may be used to treat or prevent an impulsive symptom of a substance-related or non-substance-related disorder.

In an aspect, the substance-related disorder is related to a substance selected from the group consisting of opioids, cannabis, alcohol, cocaine, tobacco and sedative, hypnotic, or anxiolytic substances.

In another embodiment, the disorder is an opioid-related disorder or cannabis-related disorder.

Also described herein are methods of treating at least one substance-induced depressive, bipolar or anxiety disorder. In an aspect, one or more compositions described herein may be administered to a patient for treatment of any substance-induced depressive, bipolar or anxiety disorder. In certain embodiments, a single composition may be administered which contains topiramate and aripiprazole (or a derivative, congener or analogue, e.g., brexpiprazole or cariprazine). In other embodiments, multiple compositions may be administered together, each comprising one or more active ingredients.

In exemplary embodiments, the substance-induced depressive, bipolar or anxiety disorder is induced by use of opioids, alcohol, cannabis, tobacco, cocaine, or a sedative, hypnotic, or anxiolytic substance. In a yet further embodiment, the substance-induced depressive, bipolar or anxiety disorder is induced by use of opioids, cannabis, alcohol, or a sedative, hypnotic, or anxiolytic substance.

In an aspect, one or more compositions described herein may be administered to a patient for treatment or prophylaxis of a non-substance-related disorder, particularly gambling disorder. In certain embodiments, a single composition may be administered which contains topiramate and aripiprazole (or a derivative, congener or analogue, e.g., brexpiprazole or cariprazine). In other embodiments, multiple compositions may be administered together, each comprising one or more active ingredients.

The administration form of compositions described herein is not particularly limited. Topiramate and aripiprazole (and/or pharmaceutically acceptable salts or a derivative, congener or analogue [e.g., brexpiprazole or cariprazine] thereof) need only be combined upon administration. Examples of such methods of administration form include but are not limited to (1) administration of a single composition or dosage form obtained by simultaneously formulating topiramate or a pharmaceutically acceptable salt thereof with aripiprazole or pharmaceutically acceptable salts or a derivative, congener or analogue (e.g. brexpiprazole or cariprazine) thereof, (2) simultaneous administration of two kinds of preparations obtained by separately formulating topiramate or a pharmaceutically acceptable salt thereof and aripiprazole or a pharmaceutically acceptable salt or a derivative, congener or analogue (e.g. brexpiprazole or cariprazine) thereof by the same administration route, (3) administration of two kinds of preparations obtained by separately formulating topiramate or a pharmaceutically acceptable salt thereof and aripiprazole or a pharmaceutically acceptable salt or a derivative, congener or analogue (e.g. brexpiprazole or cariprazine) thereof by the same administration route in a staggered manner (e.g., administration in the order of aripiprazole or a pharmaceutically acceptable salt thereof; topiramate or a pharmaceutically acceptable salt thereof, or administration in the reverse order), (4) simultaneous administration of two kinds of preparations obtained by separately formulating aripiprazole or a pharmaceutically acceptable salt or a derivative, congener or analogue (e.g. brexpiprazole or cariprazine) thereof and topiramate or a pharmaceutically acceptable salt thereof by different administration routes, (5) administration of one or more kinds of preparations obtained by separately formulating aripiprazole or a pharmaceutically acceptable salt or a derivative, congener or analogue (e.g. brexpiprazole or cariprazine) thereof and topiramate or a pharmaceutically acceptable salt thereof by different administration routes in a staggered manner (e.g., administration in the order of aripiprazole or a pharmaceutically acceptable salt or a derivative, congener or analogue (e.g. brexpiprazole or cariprazine) thereof; topiramate or a pharmaceutically acceptable salt thereof, or in the reverse order) and the like.

In an exemplary embodiment, aripiprazole is administered continually over a significant period of time in a daily dose of 1.5 mg aripiprazole and, in combination therewith, a daily dose of 23 mg topiramate.

In an exemplary embodiment, aripiprazole is administered continually over a significant period of time in a daily dose of 3 mg aripiprazole and, in combination therewith, a daily dose of 46 mg topiramate.

In another exemplary embodiment, aripiprazole is administered continually over a significant period of time in a daily dose of 4.5 mg aripiprazole and, in combination therewith, a daily dose of 69 mg topiramate.

In an exemplary embodiment, aripiprazole is administered continually over a significant period of time in a daily dose of 6 mg aripiprazole and, in combination therewith, a daily dose of 92 mg topiramate.

In another exemplary embodiment, aripiprazole is administered continually over a significant period of time in an escalating dose regimen of 1 mg aripiprazole per day in combination with 23 mg topiramate per day for one week, followed by 3 mg aripiprazole per day in combination with 46 mg topiramate per day, and followed, if there is an insufficient response, by an increase of 1.5 mg aripiprazole per day in combination with an increase of 23 mg topiramate per day every two weeks up to a maximum dose of 6 mg aripiprazole per day in combination with 92 mg topiramate per day.

Additionally, it is envisioned that brexpiprazole or cariprazine may be used in place of, or in addition to, at least one of the active pharmaceutical ingredients mentioned herein. That is, embodiments comprising any of aripiprazole, brexpiprazole, cariprazine and/or topiramate are contemplated.

Additionally, two-active embodiments are considered to be effective for treating one or more substance use disorders and/or one or more substance-induced depressive or anxiety disorder. Said two-active compositions contain as active pharmaceutical ingredients aripiprazole and topiramate, brexpiprazole and topiramate, cariprazine and topiramate, aripiprazole and brexpiprazole, or aripiprazole and cariprazine.

Although embodiments of the present disclosure have been described in terms of specific exemplary embodiments and examples, it will be appreciated that the embodiments disclosed herein are for illustrative purposes only and various modifications and alterations might be made by those skilled in the art without departing from the spirit and scope of the disclosure as set forth in the following claims.

Non-limiting examples of the presently disclosed inventive formulations are provided herein.

### EXAMPLES

### Example 1

Treatment for Opioid Use Disorder, on Maintenance Therapy (Co-Administration with Buprenorphine)

A patient with severe opioid use disorder characterized by a 10-year history of nearly continuous intravenous heroin use, multiple residential, partial hospitalization, and intensive outpatient treatment episodes, and chronic health problems including hepatitis C, frequent cutaneous abscesses, and two episodes of endocarditis undergoes sublingual buprenorphine/naloxone induction and is titrated to a maintenance dose of 12 mg daily. Despite this regimen and participation in intensive outpatient treatment and 12-step community-based support groups, the patient frequently relapses to intravenous heroin use and is unable to maintain more than two weeks of continuous abstinence from heroin. The patient's PHQ-9 score is 19 (moderately severe depression) and he reports intermittent acute episodes of drug craving which typically precede the relapses. The composition is added to the patient's buprenorphine/naloxone regimen and titrated to a dose of 3 mg aripiprazole IR/46 mg topiramate ER qAM over 1 week. Two weeks after the addition of aripiprazole/topiramate to his regimen, the patient's PHQ-9 score drops to 8 (mild depression) and he reports substantially diminished intensity and frequency of drug cravings. He is maintained on the combined regimen of buprenorphine/naloxone and aripiprazole/topiramate and subsequently establishes long-term abstinence from heroin.

### Example 2

### Treatment for Opioid Use Disorder (Prescribed Opioids)

A patient who was treated with oxycodone in the context of protracted postoperative pain status-post right hip replacement subsequently develops moderate to severe opioid use disorder, continuing her opioid use for two years after the resolution of postoperative pain and obtaining prescriptions for oxycodone, hydrocodone, and transdermal fentanyl from various physicians. The patient's substance use history and psychiatric history is otherwise unremarkable. After undergoing ambulatory opioid detoxification with clonidine, methocarbamol, gabapentin, and loperamide, the patient remains abstinent from opioids for two weeks but subsequently relapses, uses opioids for three months, and then returns to her physician for another detoxification which is completed successfully. The patient remains abstinent for 1 week post detoxification and then relapses to opioid use again. This cycle occurs twice more over the course of a year. After the fourth detoxification, the patient is referred to an addiction medicine specialist who initiates the composition which is titrated to a daily dose aripiprazole 3 mg IR/topiramate 46 mg ER. The patient is maintained on this dose of aripiprazole/topiramate and remained abstinent for greater than 12 months.

### Example 3

Prevention of Opioid Use Disorder (Prescribed Opioids) A 25-year-old male patient with a past psychiatric history significant for major depressive disorder and a family history of alcohol use disorder sustains bilateral femur fractures and extensive soft tissue injury to his lower extremities in a motor vehicle accident. He requires multiple surgeries over the course of 18 months and over this time period is continuously prescribed hydrocodone/acetaminophen 7.5 mg/325 mg tablets, one tablet PO q6 hours as needed for injury-related and post-operative pain. During the first three weeks after the accident, the patient averages 4 tabs per 24-hour period with a subsequent decrease in frequency to 2 tabs per 24 hours over the next three weeks. He undergoes several additional surgeries over the next 4 months with transient increases in hydrocodone/acetaminophen use in the first week after each surgery followed by a return to 2 tabs per 24-hour period. After 6 months of treatment, the patient's hydrocodone/acetaminophen use begins to increase despite good surgical outcomes and significant improvement in his injuries and he begins to request early refills. When asked about the increased use, the patient reports that in addition to reducing his physician pain, the hydrocodone/acetaminophen reduces his symptoms of depression and that he often finds himself looking forward to the next dose. He further reports that he sometimes takes more doses than he initially intended and that he has been reducing the amount of time he spends with friends because he prefers staying home and taking the hydrocodone/acetaminophen. Given this report and the patient's risk factors for an addictive disorder, his physician initiates the composition which is titrated to a daily dose of aripiprazole 3 mg IR/topiramate 46 mg ER. After two weeks on this dose, the patient's hydrocodone/acetaminophen use decreases to 1 tab per 24-hour period. He reports that this amount adequately controls his physical pain and that he no longer finds himself preoccupied with the hydrocodone/acetaminophen between doses or taking more than he intended. He also reports that he has stopped limiting time with his friends in order to take more hydrocodone/acetaminophen.

### Example 4

Treatment for Opioid Use Disorder, on Maintenance Therapy (Co-Administration with Long-Acting Depot Naltrexone)

After completion of a sub-acute detox program, a 32-year-old female patient with a history severe opioid use disorder is evaluated by an internist boarded in addiction medicine who recommends pharmacotherapy in addition to psychotherapy and participation in community-based treatment such as Narcotics Anonymous or SMART Recovery. After the physician reviews the relative risks and benefits of both buprenorphine/naloxone and long-acting depot naltrexone with the patient, she decides to initiate long acting depot naltrexone. After one month, the patient returns to the physician for her next injection of naltrexone. She reports that she has remained abstinent from opioids for the duration of the month but admits to significant cravings for opioids which have brought her to the brink of relapse several times. She reports that knowing the naltrexone will substantially block the effect of opioids were she to use was the reason she remained abstinent, and she is ambivalent about receiving the next month's injection of naltrexone. After discussion with the physician, the patient decides to receive the injection. She remains abstinent for the next month but does not return for her follow up appointment. She relapses to opioid use 6 weeks after the last injection and suffers a nearly fatal overdose after using her previously typical dose due to the absence of any opioid tolerance after two months of depot naltrexone. She returns to the physician requesting that the depot naltrexone be re-initiated. The physician re-initiates the depot naltrexone per the standard protocol and also initiates the composition which is titrated to a daily dose of aripiprazole 3 mg IR/topiramate 46 mg ER. On follow up one month later, the patient denies opioid cravings and requests continuation of the current regimen.

1. A pharmaceutical composition comprising:
a first active pharmaceutical ingredient which is aripiprazole or a derivative, congener, or an analogue, or a pharmaceutically acceptable salt thereof, wherein the first active pharmaceutical ingredient is present in an amount of 0.5 to 12 mg, and
a second active pharmaceutical ingredient which is topiramate or a pharmaceutically acceptable salt thereof, wherein the second active pharmaceutical ingredient is present in an amount of 5 to 150 mg;
wherein the pharmaceutical composition is a single dosage form in the form of a bilayer tablet; and
wherein the bilayer tablet comprises an immediate release drug layer contains said aripiprazole or a derivative, congener, or an analogue, or a pharmaceutically acceptable salt thereof.

2. The pharmaceutical composition according to claim 1, wherein the first active pharmaceutical ingredient is aripiprazole.

3. The pharmaceutical composition according to claim 1, further comprising a controlled release polymer selected from the group consisting of delayed release polymers and extended-release polymers.

4. (canceled)

5. The pharmaceutical composition according to claim 1, wherein the first and second active pharmaceutical ingredients are present in a ratio [first active pharmaceutical ingredient]:[second active pharmaceutical ingredient] of 1.5:23.

6. The pharmaceutical composition according to claim 3, wherein the controlled release polymer layer comprises one or more polymers selected from the group consisting of EUDRAGIT® NM 30D, ethylcellulose, polyvinylpyrrolidone, polylactides (PLA), polyglycolides (PGA), poly(lactide-co-glycolides) (PLGA), polyanhydrides, and polyorthoesters.

7. The pharmaceutical composition according to claim 1, wherein the bilayer tablet further comprises an one or more extended-release drug layers.

8. The pharmaceutical composition according to claim 1, wherein the bilayer tablet further comprises one or more delayed release drug layers.

9. The pharmaceutical composition according to claim 7, wherein the extended-release drug layer contains topiramate or a pharmaceutically acceptable salt thereof.

10. The pharmaceutical composition according to claim 8, wherein the delayed release drug layer contains topiramate or a pharmaceutically acceptable salt thereof.

11. The pharmaceutical composition according to claim 1, wherein the first active pharmaceutical ingredient comprises brexpiprazole and/or cariprazine.

12. A method of treatment or prophylaxis of substance-related and/or addictive disorders, and/or substance-induced depressive disorders, bipolar and related disorders, or anxiety disorders, comprising administering to a patient: a first active pharmaceutical ingredient which is aripiprazole, or a derivative, congener, or an analogue, or a pharmaceutically acceptable salt thereof; and a second active pharmaceutical ingredient which is topiramate or a pharmaceutically acceptable salt thereof.

13. The method of claim 12, wherein the substance-related and/or addictive disorder is selected from the group consisting of opioid-related disorders, alcohol-related disorders, cannabis-related disorders, tobacco-related disorders, hallucinogen-related disorders, stimulant-related disorders, and sedative-, hypnotic-, anxiolytic-related disorders and gambling-related disorder; and/or
wherein the substance-related disorder is selected from the group of substance use disorders consisting of opioid use disorder, alcohol use disorder, cannabis use disorder, tobacco use disorder, stimulant use disorder, hallucinogen use disorder, and sedative, hypnotic, and anxiolytic use disorder; and/or
wherein the substance-related and/or addictive disorder is selected from the group of substance-induced disorders consisting of 1) opioid-induced depressive disorder, cannabis-induced depressive disorder, alcohol-induced depressive disorder, stimulant-induced depressive disorder, sedative-, hypnotic-, or anxiolytic-induced depressive disorder, alcohol-induced depressive disorder, and gambling-induced depressive disorder, 2) opioid-induced bipolar and related disorder, cannabis-induced bipolar and related disorder, alcohol-induced bipolar and related disorder, stimulant-induced bipolar and related disorder, sedative-, hypnotic-, or anxiolytic-induced bipolar and related disorders, alcohol-induced bipolar and related disorder, and 3) opioid-induced anxiety disorder, cannabis-induced anxiety disorder, stimulant-induced anxiety disorder, sedative-, hypnotic-, or anxiolytic-induced anxiety disorder, and alcohol-induced anxiety disorder; and/or
wherein the substance-related and/or addictive disorder is a depressive disorder, bipolar and related disorder, or anxiety disorder associated with substance-induced intoxication or withdrawal, wherein the substance is selected from the group consisting of opioids, alcohol, cannabis, tobacco, stimulants, hallucinogens, sedatives, hypnotics, and anxiolytics; and/or
wherein the substance related disorder is opioid use disorder and/or the substance-induced disorder is opioid-induced depressive disorder, bipolar and related disorder, or anxiety disorder and an opioid receptor ligand is concurrently administered or used illicitly.

14. The method of claim 12, wherein treatment or prophylaxis for substance-related and/or addictive disorders, and/or substance-induced depressive disorders, bipolar and related disorders, or anxiety disorders, results in an increased retention in treatment and/or a reduction in relapse after treatment or prevents the occurrence of such disorders and/or obviates the need for additional treatments or therapies; and/or
wherein treatment initiation is concurrent with substance use, intoxication, or withdrawal.

15. The method of claim 12, wherein naltrexone has been previously administered, or is currently being administered, and/or has resulted in an adverse event or side-effect.

16. The method of claim 12, wherein the substance related disorder is opioid use disorder and/or the substance-induced disorder is opioid-induced depressive disorder, bipolar and related disorder, or anxiety disorder and treatment with an opioid receptor ligand (agonist, partial agonist, or antagonist) has resulted in inadequate response to treatment, and/or the administered opioid receptor ligand should or must be decreased or stopped.

17. The method of claim 12, wherein the method results in prophylaxis preventing opioid use disorder when opioid medications are administered for analgesia; and/or
wherein prophylaxis prevents anxiolytic use disorder when anxiolytics are administered for treatment of anxiety disorders.

18. The method of claim 12, wherein treatment or prophylaxis for substance-related and/or addictive disorders, and/or substance-induced depressive disorders, bipolar and related disorders, or anxiety disorders is effective in treating or preventing the disorders at steady state aripiprazole plasma levels of less than or equal to 70 ng/ml and topiramate plasma levels of less than or equal to 6 micrograms/ml at which 1) aripiprazole high-affinity targets dopamine D2, D2L, D3 and serotonin 5HT2B, 5-HT1A are preferentially bound by aripiprazole compared to low affinity targets, 2) the D2 receptor occupancy is reduced compared to occupancy at plasma levels required for treatment of current FDA approved therapeutic indications and 3) topiramate plasma levels are below those required for treatment of current FDA approved therapeutic indications.

19. The method of claim 12, wherein treatment or prophylaxis for substance-related and/or addictive disorders, and/or substance-induced depressive disorders, bipolar and related disorders, or anxiety disorders is effective in treating or preventing the disorders while minimizing drug-induced akathisia, extrapyramidal symptoms, nausea, weight gain, sedation or difficulty in word-finding.

20. The method of claim 12, wherein aripiprazole or a derivative, congener, or an analogue, or a pharmaceutically acceptable salt thereof is administered in an amount of 0.5 mg to 12 mg, and topiramate or a pharmaceutically acceptable salt thereof is administered in an amount of 5 mg to 150 mg; or
wherein aripiprazole or a derivative, congener, or an analogue, or a pharmaceutically acceptable salt thereof is administered continually over a significant period of time in a daily dose selected from any one of 1.5, 3, 4.5, and 6 mg, in combination therewith, a daily dose selected from any one of 23, 46, 69, and 92 mg of topiramate or a pharmaceutically acceptable salt thereof; or
wherein aripiprazole or a derivative, congener, or an analogue, or a pharmaceutically acceptable salt thereof is administered continually over a significant period of time in an escalating dose regimen of 1.5 mg aripiprazole per day in combination with 23 mg topiramate per day for one week, followed by 3 mg aripiprazole per day in combination with 46 mg topiramate per day, and followed, if there is an insufficient response, by an increase of 1.5 mg aripiprazole per day in combination with an increase of 23 mg topiramate per day every two weeks up to a maximum dose of 6 mg aripiprazole per day in combination with 92 mg topiramate per day.

21. (canceled)
